(19) United States
(12) Patent Application Publication
(10) Pub. No.: US 2009/0252717 A1
(43) Pub. Date: Oct. 8, 2009

(54) Compositions and Methods for Treating Polyglutamine-Expansion Neurodegenerative Diseases

(76) Inventors: Scott Thomas Brady, Chicago, IL (US); Gerardo Andres Morfini, Chicago, IL (US)

Correspondence Address: LICATA & TYRRELL P.C., 66 E. MAIN STREET, MARLTON, NJ, 08053, US

(21) Appl. No.: 12/302,349

(22) PCT Filed: May 29, 2007
(86) PCT No.: PCT/US2007/069847

§ 371 Date: Dec. 12, 2008

Related U.S. Application Data
(60) Provisional application No. 60/809,099, filed on May 26, 2006.

(51) Int. Cl. A61K 38/45; G01N 33/53; A61P 25/28

(52) U.S. Cl. 424/94.5; 435/7.1

(57) ABSTRACT

The invention relates to methods for stimulating fast axonal transport in polyglutamine expansion diseases and treating polyglutamine expansion diseases by inhibiting SAPK-dependent phosphorylation of kinesin. The present invention also provides methods for identifying agents which inhibit the phosphorylation of the kinesin, as well as methods for monitoring treatment of a polyglutamine expansion disease based on the phosphorylation of serine 176 of kinesin-1A or kinesin-1C, or serine 175 of kinesin-1B.

This application claims benefit of U.S. Provisional Patent Application Ser. No. 60/809,099, filed May 26, 2006, the content of which is incorporated herein by reference in its entirety.

### INTRODUCTION

This invention was made in the course of research sponsored by the National Institutes of Health (NIH grant Nos. NS23868, NS23320, NS41170, and NS43408). The U.S. government has certain rights in this invention.

### BACKGROUND OF THE INVENTION

Polyglutamine-expansion (PolyQ) diseases encompass a group of heterogeneous adult-onset neurodegenerative diseases caused by expansion of a CAG repeat, which results in extended polyQ tracts (Zoghbi & Orr (2000) Ann. Rev. Neurosci. 23:217-247). Remarkably, pathology is restricted to neurons, although mutant genes are often ubiquitously expressed. PolyQ diseases typically progress as dying-back neuropathies (Zoghbi & Orr (2000) supra). Among polyQ diseases, X-linked spinal and bulbar muscular atrophy (SBMA, Kennedy's disease) involve expansion of the polyQ stretch in the androgen receptor. The CAG repeat in the androgen receptor gene expands from 5-34 triplets in normal individuals (i.e., wild-type androgen receptor) to 40-66 (polyQ-AR) in SBMA patients (Brooks & Fischbeck (1995) Trends Neurosci. 18:459-461). Remarkably, patients with androgen resistance syndromes due to loss of androgen receptor function do not show neurodegeneration, suggesting that the neuropathological phenotype of SBMA is due to a toxic gain of function associated with expanded polyQ in the androgen receptor protein, rather than defective androgen receptor function (Brooks & Fischbeck (1995) supra). SBMA patients exhibit adult-onset proximal muscle weakness, muscle flaccidity and atrophy. These defects eventually lead to dysarthria, dysphagia and death. No effective treatments are currently available, and pathogenic mechanisms for SBMA remain unclear.

Despite the wide cellular distribution of the androgen receptor protein (Wilson & McPhaul (1996) Mol. Cell Endocrinol. 120:51-7), SBMA is a lower motor neuron disease (Brooks & Fischbeck (1995) supra). This suggests that a cellular process particularly critical for proper function and survival of motor neurons is selectively altered by polyQ-AR (Morfini, et al. (2005) Trends Mol. Med. 11:64-70). Motor neurons affected in SBMA include some of the largest (up to 5000× the volume of a typical neuron) and longest (>1 meter long in some cases) neurons in humans. These characteristics renders neuronal cells particularly vulnerable to alterations in fast axonal transport mechanisms (Morfini, et al. (2005) supra). Fast axonal transport deficits were long predicted to produce neurological defects, but recent genetic evidence provides proof of principle (Morfini, et al. (2005) supra). Several studies link specific neurodegenerative diseases to mutations in microtubule (MT)-based motor proteins of kinesin and dynein superfamilies (Hirokawa & Takemura (2003) Trends Biochem. Sci. 28:558-65; Mandelkow & Mandelkow (2002) Trends Cell Biol. 12:585-91). For example, mutations in specific cytoplasmic dynein subunits result in neuronal dysfunction (Hafezparast, et al. (2003) Science 300:808-12). Remarkably, several mutations selectively affect motor neurons. Moreover, dominant partial loss-of-function mutations in one out of three kinesin-1-heavy chain genes (kinesin-1a, KIF5a) causes an autosomal dominant form of hereditary spastic paraplegia (Reid, et al. (2002) Am. J. Hum. Genet. 71:1189-1194), a disease that also affects lower motor neurons. This latter finding demonstrates that a 50% reduction in function of a single kinesin-1 motor isoform is sufficient to cause late-onset neurodegenerative disease (Reid, et al. (2002) supra).

Consistent with these observations, reports have suggested that pathogenic polyQ proteins inhibit fast axonal transport in several polyQ diseases, including SBMA and Huntington's disease (Szebenyi, et al. (2003) Neuron 40:41-52; Gunawardena, et al. (2003) Neuron 40:25-40; Lee, et al. (2004) Proc. Natl. Acad. Sci. USA 101:3224-9; Gauthier, et al. (2004) Cell 118:127-38). Vesicle motility assays in extruded squid axoplasm showed that subnanomolar levels of soluble, non-aggregated polyQ-AR or huntingtin inhibit fast axonal transport in a transcription-independent manner (Szebenyi, et al. (2003) supra). Further, neuronal cell lines stably transfected with polyQ-AR display significantly shorter neuritic processes than wild-type androgen receptor transfected ones (Szebenyi, et al. (2003) supra), a phenotype consistent with reductions in kinesin-based motility (Amaratunga, et al. (1993) J. Biol. Chem. 268:17427-17430; Feiguin, et al. (1994) J. Cell Biol. 127:1021-1039). Given that very low polyQ-AR levels (<1 nM) inhibit fast axonal transport, the pathogenic proteins were proposed to alter enzymatic activities involved in fast axonal transport regulation (Morfini, et al. (2005) supra; Szebenyi, et al. (2003) supra).

Changes in kinesin-1 function in response to inflammatory cytokines have been suggested for some inflammatory and degenerative brain diseases. For example, Stagi ((2005) PhD Thesis, The Georg-August University Göttingen) teaches that TNF-alpha induced detachment of the heavy chain kinesin family-5B (KIF5B) protein from tubulin in axons is dependent on JNK, wherein inhibition of axonal transport by TNF is mediated via JNK phosphorylation.

Further, studies on in vivo function and regulation of motors show that phosphorylation is a major regulatory mechanism for fast axonal transport (Morfini, et al. (2001) Dev. Neurosci. 23:364-376; Morfini, et al. (2002) EMBO J. 23:281-293). Multiple regulatory pathways for fast axonal transport have been described involving several protein kinase and phosphatase activities, which directly or indirectly modify molecular motors and affect their function (Morfini, et al. (2005) supra; Morfini, et al. (2002) Neuromol. Med. 2:89-99). It has been suggested that even modest alterations in kinase-dependent regulatory pathways for fast axonal transport can lead to neuropathy (Morfini, et al. (2002) supra). For example, GSK-3 phosphorylates kinesin-1 and inhibits kinesin-based motility (Morfini, et al. (2002) supra) and mutations in the Familial Alzheimer's disease-related protein presenilin-1 lead to increased GSK-3 activity with a concurrent decrease in kinesin-based motility (Pigino, et al. (2003) J. Neurosci. 23:4499-4508). Moreover, several independent reports indicate that kinase activities are deregulated in SBMA. For example, changes in neurofilament protein phosphorylation are reported in an SBMA animal model (Chevalier-Larsen, et al. (2004) J. Neurosci. 24:4778-86). Further, increased activity of selected MAPK family members was reported in SBMA cellular models (Cowan, et al. (2003) Hum. Mol. Genet. 12:1377-91; LaFevre-Bernt, et al. (2003) J. Biol. Chem. 278:34918-24). Moreover, Apostol et al. ((2006) Hum. Mol. Gen. 15(2):273-285) report that a JNK-specific inhibitor can rescue photoreceptor neurodegeneration in vivo in a Drosophila model of Huntington's disease. In this regard, U.S. Pat. Nos. 6,288,089, 6,811,992 and 7,195,894; and U.S. Patent Application Nos. 20030148395 and 2002058245 suggest the use of JNK or MLK inhibitors for the treatment of neurological conditions such as Huntington's disease based on findings that indicate that such kinases mediate cellular apoptosis in such diseases. However, the specific pathogenic target for these kinases was not identified, and the relationship of changes in kinase activity to pathogenesis was uncertain.

Significantly, diseases such as Huntington's disease progress as dying back neuropathies in which neurological symptoms occur as a result of losing synaptic connectivity and function. Neuronal cell death is a late event that does not correlate with either symptoms or death in patients and animal models of neurodegeneration (Chiesa, et al. (2005) Proc. Natl. Acad. Sci. USA 102:238-243; Waldmeier, et al. (2006) Biochem. Pharmacol. 72:1197-1206). Thus, although anti-apoptotic agents might help preserve neuronal cell bodies, these surviving neurons would likely bear dysfunctional axons and synapses, being unable to maintain appropriate connections or to sustain neurotransmission. The recent clinical failures in Parkinson's disease (PD) using apoptosis-inhibitors underline the need for a paradigm shift in drug discovery in neurodegenerative diseases.

### SUMMARY OF THE INVENTION

The present invention is a method for restoring fast axonal transport in a cell which expresses a polyglutamine-expanded polypeptide, by contacting the cell with an effective amount of one or more agents which inhibit stress-activated protein kinase (SAPK)-dependent phosphorylation of kinesin. In particular embodiments, the kinesin is kinesin-1 and the kinesin-1 is phosphorylated at serine 176 of SEQ ID NO:1 or SEQ ID NO:7, or serine 175 of SEQ ID NO:4. In other embodiments, the SAPK is MLK3 or JNK3. In still further embodiments, the polyglutamine-expanded polypeptide is Huntingtin or androgen receptor.

The present invention is also a method for treating a polyglutamine expansion disease by administering to a subject with a polyglutamine expansion disease an effective amount of an agent which inhibits SAPK-dependent phosphorylation of a kinesin thereby treating the polyglutamine expansion disease. In particular embodiments, the kinesin is kinesin-1 and the kinesin-1 is phosphorylated at serine 176 of SEQ ID NO:1 or SEQ ID NO:7, or serine 175 of SEQ ID NO:4. In other embodiments, the SAPK is MLK3 or JNK3. In still further embodiments, the polyglutamine expansion disease is Huntington's disease, or spinal and bulbar muscular atrophy.

The present invention further provides a method for identifying an agent for treating a polyglutamine expansion disease. This method involves contacting a SAPK with a test agent in the presence of a kinesin, or substrate fragment thereof, and determining whether the test agent inhibits the phosphorylation of the kinesin or substrate fragment by the SAPK thereby identifying an agent for treating a polyglutamine expansion disease. In particular embodiments, the kinesin is kinesin-1 and the kinesin-1 is phosphorylated at serine 176 of SEQ ID NO:1 or SEQ ID NO:7, or serine 175 of SEQ ID NO:4. In other embodiments, the SAPK is MLK3 or JNK3.

The present invention also embraces a method for monitoring treatment of a polyglutamine expansion disease by determining, in a biological sample from a subject receiving therapy for a polyglutamine expansion disease, the phosphorylation state of kinesin-1, wherein a decrease in the phosphorylation of kinesin-1 after receiving therapy is indicative of treatment of the polyglutamine expansion disease.

### BRIEF DESCRIPTION OF THE DRAWINGS

FIG. 1 shows that polyQ-AR alters axonal kinase activities and increases kinesin-1 phosphorylation. Quantitative analysis of kinesin phosphorylation indicates a 50% increase in net KHC phosphorylation for cells expressing polyQ-AR relative to wild-type androgen receptor (WT AR)-expressing cells. KLC phosphorylation did not significantly change between these cell lines.

FIG. 2 shows that a SAPK inhibitor reverses the inhibitory effect of polyQ-AR expression on neurite outgrowth. SH-SY5Y cells treated with retinoic acid and BDNF in the presence of wild-type androgen receptor withdraw from the cell cycle, extend neurites and begin to express neuronal markers. SH-SY5Y cells expressing polyQ-AR also withdraw from the cell cycle, but fail to extend neurites. FIG. 2A shows the quantitation of total neurite lengths for SH-SY5Y cells after 3 days BDNF treatment. Note that untreated polyQ-AR cells are significantly smaller than cells expressing wild-type androgen receptor (WT AR) (p<0.001). However, polyQ-AR cells increased in length with 10 μM SB203580 and were indistinguishable from wild-type androgen receptor cells treated with 10 μM SB203580. Wild-type androgen receptor and polyQ-AR cells in the presence of 20 μM SB203580 were indistinguishable from each other and from untreated wild-type androgen receptor cells. FIG. 2B is a histogram showing distribution of cell shapes for each condition to illustrate a shift in cell shape with increasing concentrations of SB203580. Note that the majority of cells in untreated polyQ-AR cultures have short neurites, but wild-type androgen receptor (WT AR) cultures are enriched in cells bearing longer neurites. Addition of 10 μM or 20 μM SB203580 to the media induced a significant increase in the number of polyQ-AR cells extending long neurites. PolyQ-AR cells in the presence of 20 μM SB203580 were indistinguishable from untreated wild-type androgen receptor cells and from wild-type androgen receptor cells treated with 20 μM SB203580. Thus, inhibition of SAPK activities with SB203580 reverses inhibition of neurite formation by polyQ-AR.

FIG. 3 shows that active JNK3 inhibits fast axonal transport. The effects of active, recombinant JNK1, JNK2 and JNK3 were evaluated using vesicle motility assays in isolated squid axoplasm. Box plots of mean anterograde (A) and retrograde (R) fast axonal transport rates in axoplasms perfused with JNK1, JNK2 and JNK3. Data represent pooled measurements taken between 30 and 50 minutes of observation.

FIG. 4 shows that the phosphorylated serine of kinesin-1 (underlined; serine 176 of kinesin-1A and kinesin-1C, serine 175 of kinesin-1B) is conserved in squid, mice and human KHC sequences.

FIG. 5 shows the treatment of kinesin-1 heavy chain with JNK3 kinase inhibits the binding of kinesin-1 to microtubules. The graph depicts the marked decrease in the ratio of microtubule-associated (P) versus soluble (S) kinesin-1 for JNK-phosphorylated kinesin-1, versus non-phosphorylated kinesin-1.

### DETAILED DESCRIPTION OF THE INVENTION

Mutations in proteins as diverse as Huntingtin (Htt) and androgen receptor (AR), lead to selective neuronal degeneration. This indicates the existence of multiple, distinct pathways which converge on a common target. It has now been found that polyglutamine (polyQ) expansion polypeptide-induced fast axonal transport inhibition occurs via a pathway involving activation of stress-associated protein kinases (SAPKs), specifically Mixed Lineage Kinase 3 (MLK3) and cJun N-terminal kinase 3 (JNK3). In particular, it has been demonstrated that polyQ expansion polypeptide-induced fast axonal transport inhibition involves phosphorylation of kinesin-1 heavy chain (KHC) subunits by JNK and inhibition of kinesin-1 function. Furthermore, polyQ-AR and polyQ-Htt, but not wild-type androgen receptor or wild-type Htt expression in cells resulted in increased JNK activity, increased kinesin-1 heavy chain (KHC) phosphorylation at a specific serine residue involved in the interaction of kinesin-1 with microtubules, and inhibition of kinesin-1 binding to microtubules. Moreover, JNK and MLK kinase inhibitors prevented the effects of polyQ expansion polypeptide-induced inhibition on fast axonal transport in squid axoplasm and cellular models of Huntington's disease and Spinal Bulbar Muscular Atrophy. The basis for fast axonal transport inhibition by polyglutamine-expanded polypeptides results from increased binding of these mutant polypeptides to MLK, compared to normal, nonpathogenic proteins, wherein said increased binding disrupts the previously described autoinhibitory intramolecular interaction in MLK (Zhang & Gallo (2001) J. Biol. Chem. 276:45598-45603).

In contrast to the teachings of the prior art, the data provided herein indicates that loss of synaptic function and the consequent distal axonopathy, rather than cell death, represent the source for neurological problems in polyglutamine expansion diseases. As such, the identified correlation of JNK and MLK kinase activation, kinesin-1 phosphorylation, and fast axonal transport inhibition to SBMA and Huntington's Disease pathogenesis provides a novel therapeutic target to limit, delay or prevent progressive neurodegeneration in polyglutamine expansion diseases.

Accordingly, the present invention relates to a method for restoring fast axonal transport defects in a cell which expresses a polyglutamine-expanded polypeptide by inhibiting stress-activated protein kinase (SAPK)-dependent phosphorylation of kinesins. For the purposes of the present invention, fast axonal transport is defined as kinesin- and dynein-mediated movement of mitochondria, lipids, synaptic vesicles, proteins, and other membrane-bound organelles and cellular components to and from a neuron's cell body through the axonal cytoplasm (the axoplasm) (Morfini, et al. (2006) In: Basic Neurochemistry (Ed. Siegel, et al.) pp. 485-502). Axonal transport is also responsible for moving molecules destined for degradation from the axon to lysosomes to be broken down. Axonal transport can be divided into anterograde and retrograde categories. Anterograde transport carries products like membrane-bound organelles, cytoskeletal elements and soluble substances away from the cell body towards the synapse and other axonal subdomains (Oztas (2003) Neuroanatomy 2:2-5). Retrograde transport sends chemical messages and endocytosis products headed to endolysosomes from the axon back to the cell. In accordance with the disclosure provided herein, agents that inhibit SAPK-mediated phosphorylation of kinesins can stimulate both anterograde as well as retrograde transport, in particular when said transport has been inhibited by a polyglutamine-expanded polypeptide.

Cells which express a polyglutamine-expanded polypeptide include cells, in particular neurons, from a subject with a polyglutamine expansion disease as well as neurons from a model system (e.g., an animal model or cell line as disclosed herein) of a polyglutamine expansion disease. In this regard, the cells can undergo pathogenesis, because of expressing the polyglutamine-expanded polypeptide or alternatively, the cells can be induced to express the polyglutamine-expanded polypeptide by recombinant approaches. Such recombinant expression of proteins in cells is conventional in the art and any suitable method can be employed. In some embodiments, cells of the present invention are isolated (e.g., grown in vitro). In other embodiments, cells of the instant method are in vivo.

A number of naturally occurring polypeptides have uninterrupted tracts of glutamine residues, encoded by the CAG triplet repeats. It is now known that the expansion of the length of these uninterrupted tracts or regions of trinucleotide repeats in polypeptides is associated with specific neurodegenerative diseases. The expansion of polyglutamine tracts in polypeptides can become pathogenic if the polyglutamine tracts expand beyond a threshold length, which for most polyglutamine expansion diseases is a length of approximately 35-40 residues. Thus, it will be understood that the number of glutamine repeats present in a polyglutamine-expanded polypeptide can vary from subject to subject but the polyglutamine-expanded polypeptide will still be considered to be a mutant polypeptide because it has an expanded polyglutamine region as compared to a normal, non-mutant polypeptide. For example, non-mutant huntingtin is a polymorphic protein encoded by DNA, which typically contains 10 to 35 copies of the CAG repeat, but a huntingtin polypeptide encoded by DNA with more than about 35 copies of CAG will have an expanded polyglutamine stretch and is considered a mutant, pathogenic huntingtin polypeptide. One of ordinary skill will be able to determine whether the number of polyglutamines in a polypeptide is a number that indicates the polypeptide is a mutant or non-mutant polyglutamine polypeptide. A mutant polyglutamine polypeptide has abnormal function and/or activity or an additional activity or function as compared to the non-mutant polyglutamine protein. These abnormal or mutant proteins of naturally occurring polypeptides are referred to herein as “polyglutamine-expanded polypeptides”.

When a threshold of glutamines within polyglutamine tracts is reached, the presence of the polyglutamine-expanded polypeptides is associated with neurodegenerative diseases such as Huntington's disease, spinocerebellar ataxias (SCAs), spinobulbar muscular atrophy (SBMA, Kennedy disease), and dentatorubropallidoluysian atrophy (DRPLA). In this regard, Huntington's disease is characterized by mutant of the huntingtin protein (Htt; GENBANK Accession No. NP_{—}002102), whereas Spinocerebellar Ataxia Type 1 (SCA1) and Spinocerebellar Ataxia Type 2 (SCA2) are characterized respectively by mutation of the ataxin-1 (ATXN1; GENBANK Accession No. NP_{—}000323) and ataxin-2 (ATXN2; GENBANK Accession No. NP_{—}002964) proteins. In spinocerebellar Ataxia Type 3 (SAC3), which is also known as Machado-Joseph disease (MJD), the ataxin-3 protein (ATXN3; GENBANK Accession Nos. NP_{—}004984 and NP_{—}109376) is mutated with characteristic expanded polyglutamine stretches. Spinocerebellar Ataxia Type 7 (SCA7) is associated with an abnormal expanded polyglutamine regions it the ataxin-7 protein (ATXN-7; GENBANK Accession No. NP_{—}000324). In spinocerebellar ataxia Type 6 (SCA6) there are polyglutamine expanses in the alpha-1A isoform of the calcium channel subunit (CACNA1A; GENBANK Accession No. NP_{—}075461). In spinobulbar muscular atrophy (SBMA), CAG repeats located in the androgen receptor gene result in abnormal polyglutamine stretches in the androgen receptor protein (AR; GENBANK Accession Nos. NP_{—}000035 and NP_{—}001011645). In DRPLA, the DRPLA gene exhibits abnormal CAG repeats and encodes mutant atrophin-1 protein (ATN1; GENBANK Accession No. NP_{—}001931), which shows expanded polyglutamine stretches characteristic of the polyglutamine expansion diseases.

Based upon the findings disclosed herein, inhibitors of SAPK, in particular JNK and MLK, find application in blocking or inhibiting the phosphorylation of kinesin thereby preventing fast axonal transport defects elicited by polyglutamine-expanded polypeptides. Because both MLK3 and JNK3 are SAPKs, MLK3 activates JNK3, and JNK3 directly phosphorylates kinesin, phosphorylation of kinesin is said to be SAPK-dependent. SAPK activities which can be inhibited include, e.g., any biochemical, cellular, or physiological property that varies with any variation in SAPK gene transcription or translation, or SAPK protein activity. An effective amount of a SAPK inhibitor, or JNK or MLK inhibitor, is an amount that measurably decreases or inhibits any property (e.g., phosphorylation) or biochemical activity possessed by the protein, e.g., a kinase activity or an ability to bind to another protein such as kinesin or a polyglutamine-expanded polypeptide. In one embodiment, the activity that is targeted by the inhibitory agent is JNK's or MLK's kinase activity. By inhibiting JNK or MLK kinase activity with an agent, kinesin phosphorylation is inhibited, and fast axonal transport is restored or preserved.

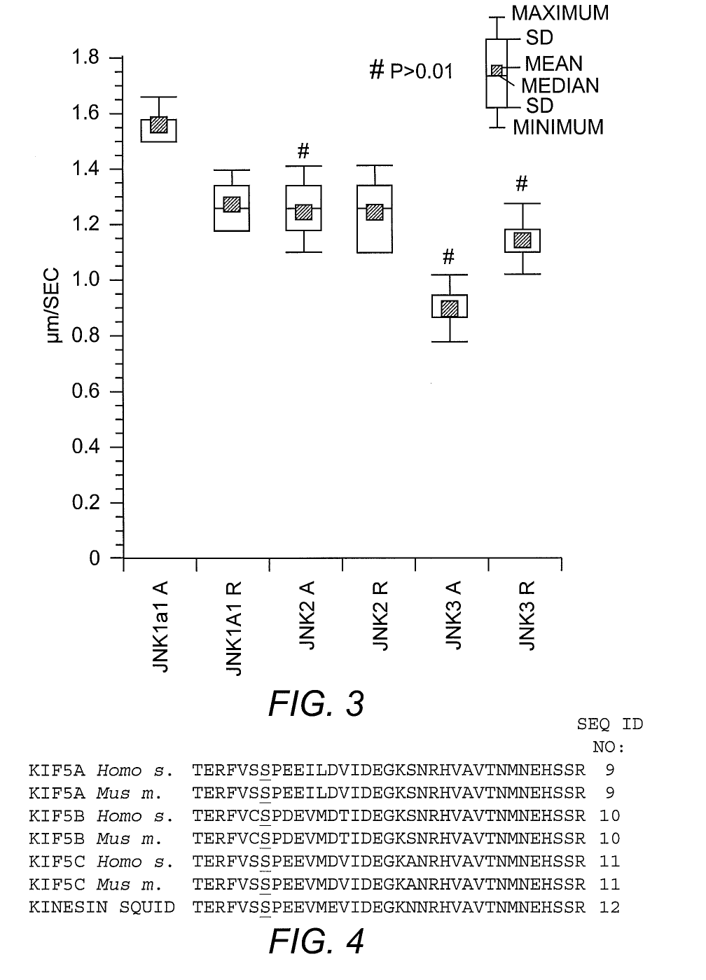

A kinesin of particular interest in accordance with the present invention is kinesin-1, specifically the heavy chain of kinesin-1. Kinesin-1 heavy chain is the most abundant kinesin in adult mammalian brain and is highly conserved across species. The protein sequences for kinesin-1 proteins are well-known in the art. Sequences for kinesin-1A (KIF5A) are found under GENBANK Accession Nos. NP_{—}004975 (Homo sapiens; SEQ ID NO:1), NP_{—}001034089 (Mus musculus; SEQ ID NO:2) and NP_{—}997688 ((Rattus norvegicus; SEQ ID NO:3). Sequences for kinesin-1B (KIF5B) are found under GENBANK Accession Nos. NP_{—}004512 (Homo sapiens; SEQ ID NO:4), NP_{—}032474 (Mus musculus; SEQ ID NO:5), and NP_{—}476550 (Rattus norvegicus; SEQ ID NO:6). Furthermore, sequences for kinesin-1C (KIF5C) are found under GENBANK Accession Nos. NP_{—}004513 (Homo sapiens; SEQ ID NO:7) and NP_{—}032475 (Mus musculus; SEQ ID NO:8). Moreover, as depicted in FIG. 4, the location of serine 176 in kinesin-1A and kinesin-1C, and serine 175 in kinesin-1B is highly conserved across species. Accordingly, particular embodiments embrace inhibiting the phosphorylation of serine 176 of SEQ ID NO:1, SEQ ID NO:2, SEQ ID NO:3, SEQ ID NO: 7, or SEQ ID NO:8; or serine 175 of SEQ ID NO:4, SEQ ID NO: 5, or SEQ ID NO:6.

In certain embodiments, the JNK inhibited includes JNK1, JNK2 and JNK3. In a particular embodiment, the JNK inhibited is JNK3. Exemplary agents which inhibit JNK include, but are not limited to, inhibitors based on the 6,7-dihydro-5H-pyrrolo[1,2-a]imidazole scaffold (e.g., ER-181304), SB203580 and SP600125.

In other embodiments, the MLK inhibited includes MLK1, MLK2 and MLK3. In a particular embodiment, the MLK inhibited is MLK3. By inhibiting MLK, the activation of JNK, and hence phosphorylation of kinesin, is inhibited thereby resulting in the stimulation, restoration or preservation of fast axonal transport. Exemplary agents which inhibit MLK include, but are not limited to, CEP-1347 and CEP11004.

Optionally, agents which inhibit SAPK-dependent (or JNK3- or MLK3-dependent) phosphorylation of kinesin for use stimulating fast axonal transport and treating polyglutamine expansion diseases can be identified in screening assays. In general, such screening assays include contacting a SAPK, e.g., JNK or MLK, with a test agent in the presence of a kinesin, or substrate fragment thereof (e.g., 10-100 amino acid residue peptide containing serine 176 of kinesin-1A or kinesin-1C or serine 175 of kinesin-1B), and determining whether the test agent inhibits the phosphorylation of the kinesin or substrate fragment by the SAPK. In some embodiments, such assays are carried out in vitro. In other embodiments, such assays are carried out in vivo.

According to in vitro aspects of the screening assay of the invention, a putative inhibitory agent is incubated in vitro in the presence of JNK and an appropriate JNK substrate (e.g., kinesin) and a phosphate donor like adenosine triphosphate (ATP), under conditions sufficient for enzymatic activity; followed by isolating the phosphorylated product. Isolated JNK proteins, including JNK1, JNK2 and JNK3, can be obtained for this, as well as other assays, by several different molecular and chromatographic methods known to those skilled in the art. The JNK polypeptides useful in the methods of the present invention are preferably wild-type whose sequence is known and readily available. For example, the human JNK3 polypeptide is described by Martin, et al. ((1996) Mol. Brain Res. 35:47-57). Other JNK proteins useful in the methods of the invention include those described in GENBANK Accession Nos. NP_{—}002744, NP_{—}620446, NP_{—}620447 and NP_{—}620448. By way of illustration, isolated JNK protein, from about 0.5 μg to about 2 μg of purified JNK, is incubated with substrate in an aqueous medium, such as a kinase buffer (containing, e.g., about 20 mM HEPES, pH 7.5, 15 mM MgCl₂, 15 mM β-glycerophosphate, 0.1 mM Na₂PO₄ and 2 mM dithiothreitol) at about 30° C. for approximately 15 minutes. Kinesin can be employed in the range of from about 1 μg to about 3 μg, and the phosphate donor, ATP, at approximately 100 μM. For detection purposes, 5 μCi of γ-³²P-ATP can be used as a co-substrate. The assay system can also include in the incubation mixture a putative inhibitory JNK agent. The reaction can be terminated by addition of Laemmeli buffer, approximately 20 μl. The addition of this buffer will also prepare the sample for product analysis. The reaction mixture can be subjected to sodium dodecylsulfate polyacrylamide gel electrophoresis (hereinafter SDS-PAGE) in order to determine the amount of phosphorylated kinesin that was formed in the reaction. The radioactivity emitted from the γ-³²P can be measured using conventional radioactivity gel detection systems, such as an X-ray film autoradiography or PHOSPHORIMAGER scan. The phosphorylated kinesin product will have a different migration rate along the gel when compared to autophosphorylated JNK and therefore will not be confused with the phosphorylating kinase. A determination can then be made concerning whether the test agent inhibited JNK's activity by comparing reaction mixtures having the agent present to reaction mixtures without addition of the compound.

Alternatively, JNK substrates, such as kinesin and ATP, can be incubated in the presence of a cellular extract containing JNK enzyme activity, including JNK1, JNK2 and JNK3. An inhibitory agent to be tested can be placed in the reaction vial along with the other reactants to examine the efficacy of the agent. The reaction and detection protocol can be conducted in the same manner as that described above for the in vitro assay without cellular extract. The cellular extract can originate from a cell or tissue culture system, or can be prepared from whole tissue employing isolation and purification protocols known to those skilled in the art.

In another embodiment, the invention pertains to contacting a cell with a putative inhibitory agent in order to screen for inhibitory agents of JNK activity, including JNK1, JNK2 and JNK3. The cell to be contacted can be of a cell or tissue culture system. The putative inhibitory agent is delivered to the cell under conditions sufficient for enzymatic activity in any of a number of ways known to those skilled in the art. If the agent is not membrane permeable, then the agent can be delivered into the cell via electroporation, or if it is a polypeptide, a nucleic acid or viral vector can be employed. If the cell has JNK present in an active form, then JNK can be stimulated by delivering to the cell SEK1, a known stimulator of JNK. If the cell lacks a JNK gene or functional JNK gene or transcript or translational product, the cell can be transfected with an operatively linked JNK gene. “Operatively linked” is intended to mean that the nucleotide sequence is linked to a regulatory sequence in a manner which allows expression of the nucleic acid sequence.

To detect the phosphorylated product, any number of methods and protocols known to those skilled in the art can be used including, but not limited to, western blot, mass spectrometric approaches, and methods for the analysis of fast axonal transport, e.g., as disclosed herein. Antibodies, both monoclonal and polyclonal, can be made against epitopes derived from the site on the JNK substrate bound to a phosphate group. A SDS-PAGE procedure can be performed on homogenized cell extracts and subsequently subjected to western blot analysis using an antibody specific for a phosphorylated JNK substrate, such as kinesin.

In another embodiment, the invention pertains to a method for screening potential inhibitory agents of JNK activity, including JNK1, JNK2 and JNK3, by administering to an animal, including mammals, the agent and determining what effect, if any, the agent has on the animal's physiological status. The animal is given an amount of test agent sufficient to allow for proper pharmacodynamic absorption and tissue distribution in the animal. Preferably, the animal used is an example of a model system mimicking the polyglutamine expansion disease of interest. However, to test the safety of the putative agent, a normal animal is preferably also subjected to the treatment. Following administration of the agent, the animal can be sacrificed and tissue sections from the brain, as well as other tissues, can be harvested and examined as above. In another embodiment, an animal model afflicted with a polyglutamine expansion disease can be administered a JNK and/or MLK inhibitor and the symptoms associated with the polyglutamine expansion disease are evaluated. Attenuation, amelioration or improvement of the polyglutamine expansion disease symptoms can be assessed, whereby improvement is indicative of the inhibitors ability to prevent and/or treat the polyglutamine expansion disease.

The methods described above can likewise be employed to identify/screen for inhibitory agents of MLK, including MLK1, MLK2 and MLK3. Appropriate MLK substrates include, but are not limited to, MKK4 and MKK7, both MAPK kinase kinases known to activate JNKs by phosphorylation at the activation loop of JNK. The MLK polypeptides useful in the methods of the present invention are preferably wild-type whose sequence is known and readily available. The human MLK3 polypeptide is described by Ing, et al. ((1994) Oncogene 9:1745-1750). Another MLK protein useful in the methods of the invention is described in GENBANK Accession No. NP_{—}002410.

The JNK and MLK proteins useful in the methods of the invention are not limited to the naturally occurring sequences described above. JNK and MLK containing substitutions, deletions, or additions can also be used, provided that those polypeptides retain at least one activity associated with the naturally occurring polypeptide and are at least 70% identical to the naturally occurring sequence. An example of a JNK or MLK that is not naturally occurring, though useful in the methods of the invention, is a JNK-gluthathione-S-transferase (JNK-GST) fusion protein. Such a protein can be produced in large quantities in bacteria and isolated. The JNK fusion protein can then be used in an in vitro kinase assay in the presence or absence of a candidate agent for treating polyglutamine expansion diseases.

Candidate agents encompass numerous chemical classes, although typically they are organic compounds. In some embodiments, the candidate agents are small organic compounds, i.e., those having a molecular weight of more than 50 yet less than about 2500, preferably less than about 1000 and, more preferably, less than about 500. Candidate agents generally include functional chemical groups necessary for structural interactions with proteins and/or nucleic acid molecules, and typically include at least an amine, carbonyl, hydroxyl or carboxyl group, preferably at least two of the functional chemical groups and more preferably at least three of the functional chemical groups. The candidate agents can have a cyclic carbon or heterocyclic structure and/or aromatic or polyaromatic structures substituted with one or more of the above-identified functional groups. Candidate agents also can be biomolecules such as peptides, proteins, antibodies, saccharides, fatty acids, sterols, isoprenoids, purines, pyrimidines, derivatives or structural analogs of the above, or combinations thereof and the like. Where the agent is a nucleic acid molecule, the agent typically is a DNA or RNA molecule, although modified nucleic acid molecules as defined herein are also contemplated.

Candidate agents are obtained from a wide variety of sources including libraries of synthetic or natural compounds. For example, numerous means are available for random and directed synthesis of a wide variety of organic compounds and biomolecules, including expression of randomized oligonucleotides, synthetic organic combinatorial libraries, phage display libraries of random peptides, and the like. Alternatively, libraries of natural compounds in the form of bacterial, fungal, plant and animal extracts are available or readily produced. Additionally, natural and synthetically produced libraries and compounds can be readily be modified through conventional chemical, physical, and biochemical means. Further, known pharmacological agents (e.g., those disclosed herein) can be subjected to directed or random chemical modifications such as acylation, alkylation, esterification, amidification, etc. to produce structural analogs of the agents.

A variety of other reagents also can be included in the screening assays disclosed herein. These include reagents such as salts, buffers, neutral proteins (e.g., albumin), detergents, etc. which may be used to facilitate optimal protein-protein binding. Such a reagent can also reduce non-specific or background interactions of the reaction components. Other reagents that improve the efficiency of the assay such as protease inhibitors, nuclease inhibitors, antimicrobial agents, and the like may also be used.

In particular embodiments, the agents of the present invention are designed to selectively inhibit a specific SAPK, e.g., JNK or MLK. Desirably, the kinase inhibitors selectively decrease a specific kinase activity in neurons and protect neurons by preserving fast axonal transport thereby allowing a broad range of clinical applications. Because JNK3 is exclusively expressed in neuronal cells, and because this JNK can be selectively attenuated, side effects in peripheral tissues will likely be negligible. A specific inhibitor of MLK or JNK should be an effective, low toxic neuroprotective drug for the treatment of a wide range of polyglutamine expansion diseases.

In this regard, the present invention also pertains to methods for the prevention or treatment of neurological conditions, specifically polyglutamine expansion neurodegenerative diseases, either through prophylatic administration prior to the occurrence of an event known to cause such diseases or therapeutic administration immediately following the event and periodically thereafter. Such prophylatic and therapeutic treatments are intended to preserve fast axonal transport and/or reduce neurodegeneration. Given the involvement of JNK and MLK in the cascade leading to inhibition of fast axonal transport, these two kinases present targets for a therapeutic regime. Accordingly, while some embodiments embrace targeting JNK or MLK, other embodiments embrace targeting both kinases of the signaling pathway. Using different kinase inhibitors with similar clinical effects will allow the development of a clinical protocol to avoid drug tolerance and provide a life-long treatment.

According to the method, a mammal, including human, is administered an effective therapeutic amount of an agent that inhibits SAPK-dependent phosphorylation of a kinesin. An effective amount for a given agent is that amount administered to achieve the desired result, for example, the inhibition of kinase activity of either JNK or MLK or both, or attenuation, amelioration of or improvement in the symptoms associated with the neurological condition.

As used herein, the term “polyglutamine expansion disease” includes Huntington's disease, spinocerebellar ataxias (e.g. SCA1, SCA2, SCA3/MJD, SCA6, SCA7, SCA17), spinobulbar muscular atrophy (SBMA, Kennedy disease), dentatorubropallidoluysian atrophy (DRPLA), and other diseases associated with proteins with expanded polyglutamine regions.

JNK or MLK inhibitors of the present invention can be administered subcutaneously, intravenously, parenterally, intraperitoneally, intradermally, intramuscularly, topically, enteral (for example, orally), rectally, nasally, buccally, vaginally, by inhalation spray, by drug pump or via an implanted reservoir in dosage formulations containing conventional non-toxic, physiologically (or pharmaceutically) acceptable carriers or vehicles.

In a specific embodiment, it may be desirable to administer the agents of the invention locally to a localized area in need of treatment; this can be achieved by, for example, and not by way of limitation, local infusion during surgery, topical application, transdermal patches, by injection, by means of a catheter, by means of a suppository, or by means of an implant, said implant being of a porous, non-porous, or gelatinous material, including membranes, such as sialastic membranes or fibers.

In a specific embodiment when it is desirable to direct the agent to the central nervous system, techniques which can opportunistically open the blood brain barrier for a time adequate to deliver the drug there through can be used. For example, a composition of 5% mannitose and water can be used. The present invention also provides pharmaceutical compositions. Such compositions include a therapeutically (or prophylactically) effective amount of the agent, and a physiologically acceptable carrier or excipient.

Suitable pharmaceutically acceptable carriers include but are not limited to water, salt solutions (for example, NaCl), alcohols, gum arabic, vegetable oils, benzyl alcohols, polyethylene glycols, glycerol, gelatin, carbohydrates such as lactose, amylose or starch, magnesium stearate, talc, silicic acid, viscous paraffin, perfume oil, fatty acid esters, hydroxymethylcellulose, polyvinyl pyrolidone, and combinations thereof. The pharmaceutical preparations can be sterilized and if desired, mixed with auxiliary agents, for example, lubricants, preservatives, stabilizers, wetting agents, emulsifiers, salts for influencing osmotic pressure, buffers, coloring, flavoring and/or aromatic substances and the like which do not deleteriously react with the active agents.

The compositions, if desired, can also contain minor amounts of wetting or emulsifying agents, or pH buffering agents. The composition can be a liquid solution, suspension, emulsion, tablet, pill, capsule, sustained release formulation, or powder. The composition can be formulated as a suppository, with traditional binders and carriers such as triglycerides. Oral formulation can include standard carriers such as pharmaceutical grades of mannitol, lactose, starch, magnesium stearate, polyvinyl pyrollidone, sodium saccharine, cellulose, magnesium carbonate, etc.

The compositions can be formulated in accordance with the routine procedure as a pharmaceutical composition adapted for intravenous administration to human beings. Typically, compositions for intravenous administration are solutions in sterile isotonic aqueous buffer. Where necessary, the composition can also include a solubilizing agent and a local anesthetic to ease pain at the site of the injection. Generally, the ingredients are supplied either separately or mixed together in unit dosage form, for example, as a dry lyophilized powder or water free concentrate in a hermetically sealed container such as an ampoule or sachette indicating the quantity of active agent. Where the composition is to be administered by infusion, it can be dispensed with an infusion bottle containing sterile pharmaceutical grade water, saline or dextrose/water. Where the composition is administered by injection, an ampoule of sterile water for injection or saline can be provided so that the ingredients may be mixed prior to administration.

For topical application, there are employed as nonsprayable forms, viscous to semi-solid or solid forms comprising a carrier compatible with topical application and having a dynamic viscosity preferably greater than water. Suitable formulations include but are not limited to solutions, suspensions, emulsions, creams, ointments, powders, enemas, lotions, sols, liniments, salves, aerosols, etc., which are, if desired, sterilized or mixed with auxiliary agents, for example, preservatives, stabilizers, wetting agents, buffers or salts for influencing osmotic pressure, etc. The drug may be incorporated into a cosmetic formulation. For topical application, also suitable are sprayable aerosol preparations wherein the active ingredient, preferably in combination with a solid or liquid inert carrier material, is packaged in a squeeze bottle or in admixture with a pressurized volatile, normally gaseous propellant, e.g., pressurized air.

The amount of agents which will be effective in the treatment of a particular disorder or condition will depend on the nature of the disorder or condition, and can be determined by standard clinical techniques. In addition, in vitro or in vivo assays can optionally be employed to help identify optimal dosage ranges. The precise dose to be employed in the formulation will also depend on the route of administration, and the seriousness of the disease or disorder, and should be decided according to the judgment of the practitioner and each patient's circumstances.

Common features of polyglutamine expansion diseases include the gradual loss of neurons through a dying back pattern of degeneration with a concomitant loss of motor and cognitive functions, but there are clinical differences in the various diseases. For example, the onset of Huntington's disease is characterized by choreic movements that result from the selective involvement of medium spiny neurons of the striatum. In contrast, the onset of SBMA, which is an X-linked disease involving a polyglutamine tract in the androgen receptor, is characterized by weakness and swallowing difficulties because motor neurons in the brain stem and spinal cord are selectively lost (Paulson (2000) Brain Pathology 10:293 299). As each of the polyglutamine expansion diseases progresses, more regions of the brain and spinal cord of the patient become involved. Therefore, prevention or treatment will include an amelioration of or improvement in one or more of these symptoms.

As used herein, the term “subject” is intended to include any mammal that may be in need of treatment with an agent of the invention. Subjects include but are not limited to, humans, non-human primates, cats, dogs, sheep, pigs, horses, cows, rodents such as mice, hamsters, and rats.

Having identified that phosphorylation of serine 176 of kinesin-1A or kinesin-1C, or serine 175 of kinesin-1B is a primary pathogenic event in polyglutamine expansion diseases, the present invention also provides to a method for monitoring or evaluating efficacy of treatment of a polyglutamine expansion disease in a subject by determining, in a biological sample from the subject, the phosphorylation state of serine 176 of kinesin-1A or kinesin-1C, or serine 175 of kinesin-1B, wherein a decrease in the amount of phosphorylated serine 176 of kinesin-1A or kinesin-1C, or serine 175 of kinesin-1B as compared to an untreated sample or control sample (e.g., a sample from the subject prior to treatment) is indicative of successful treatment of a polyglutamine expansion disease as disclosed herein. In particular embodiments, the subject is being treated with a therapeutic agent, e.g., as identified by the screening method of the invention. In another embodiment, the subject is being treated as part of a clinical trial, wherein determining the phosphorylation state of kinesin is to evaluate whether a test agent is efficacious in humans.

According to the invention, a biological sample can include cells, fluids, tissues and/or organs obtained by any means such that said cells, fluids, tissues, and/or organs are suitable for determining the phosphorylation state of serine 176 of kinesin-1A or kinesin-1C, or serine 175 of kinesin-1B. In some embodiments of the invention, the biological sample is biopsied, resected, drawn or otherwise harvested from a subject. In other embodiments of the invention, the biological sample is presented for analysis within its native in vivo context. A non-limiting example for in vivo detection is novel magnetic resonance imaging techniques (Jacobs, et al. (2001) J. Nucl. Med. 42(3):467-475; Wunderbaldinger, et al. (2000) Eur. J. Radiol. 34(3):156-165), wherein the biological sample may be identified and subjected to analysis while remaining in a living subject throughout.

The phosphorylation state of serine 176 of kinesin-1A or kinesin-1C, or serine 175 of kinesin-1B can be determined using mass spectrometry methods known in the art.

Alternatively, the phosphorylation state of serine 176 of kinesin-1A or kinesin-1C, or serine 175 of kinesin-1B can be determined using, e.g., an antibody which specifically recognizes the phosphorylation state of serine 176 of kinesin-1A (SEQ ID NO:1) or kinesin-1C (SEQ ID NO:7), or serine 175 of kinesin-1B (SEQ ID NO:4). Such an antibody may be delivered to cells in vitro or in vivo using particle bombardment (see, e.g., U.S. Pat. No. 5,836,905) or any other delivery technique known in the art.

An antibody is said to specifically recognize the phosphorylation state of kinesin-1 if it is able to discriminate between the unphosphorylated and phosphorylated forms of kinesin-1. For example, an antibody which specifically recognizes the phosphorylated state of kinesin will only bind to a kinesin-1A or kinesin 1C with a phosphorylated serine 176, or kinesin-1B with a phosphorylated serine 175 but will not bind to a kinesin-1A or kinesin 1C with an unphosphorylated serine 176 or a kinesin-1B with an unphosphorylated serine 175.

A method of using antibodies which specifically recognize the phosphorylation state of kinesin generally involves contacting a sample with said antibody and detecting the formation of an antigen-antibody complex using an immunoassay. The kinesin-1 antigen, as used herein, includes both the phosphorylated and unphosphorylated forms, however, the phosphorylated state is preferred. The conditions and time required to form the antigen-antibody complex may vary and are dependent on the sample being tested and the method of detection being used. Once non-specific interactions are removed by, for example, washing the sample, the antigen-antibody complex is detected using any one of the well-known immunoassays used to detect and/or quantitate antigens. Exemplary immunoassays which may be used in the method of the invention include, but are not limited to, enzyme-linked immunosorbent, immunodiffusion, chemiluminescent, immunofluorescent, immunohistochemical, radioimmunoassay, agglutination, complement fixation, immunoelectrophoresis, western blots, mass spectrometry, antibody array, and immunoprecipitation assays and the like which may be performed in vitro, in vivo or in situ. Such standard techniques are well-known to those of skill in the art (see, e.g., Methods in Immunodiagnosis (1980) 2^{nd} Edition, Rose and Bigazzi, eds. John Wiley & Sons; Campbell et al. (1964) Methods and Immunology, W. A. Benjamin, Inc.; Oellerich (1984) J. Clin. Chem. Clin. Biochem. 22:895-904; Harlow and Lane (1988) Antibodies: A Laboratory Manual, Cold Spring Harbor Laboratory, New York).

Antibodies of use in accordance with the present invention can be monoclonal or polyclonal. It is contemplated that such antibodies can be natural or partially or wholly synthetically produced. All fragments or derivatives thereof which maintain the ability to specifically bind to and recognize the phosphorylation state of kinesin-1 are also contemplated. The antibodies can be a member of any immunoglobulin class, including any of the classes: IgG, IgM, IgA, IgD, and IgE. Derivatives of the IgG class, however, are preferred in the present invention.

Antibody fragments can be any derivative of an antibody which is less than full-length. Preferably, the antibody fragment retains at least a significant portion of the full-length antibody's specific binding ability. Examples of antibody fragments include, but are not limited to, Fab, Fab′, F(ab′)₂, scFv, Fv, dsFv diabody, or Fd fragments. The antibody fragment may be produced by any means. For instance, the antibody fragment may be enzymatically or chemically produced by fragmentation of an intact antibody or it may be recombinantly produced from a gene encoding the partial antibody sequence. The antibody fragment may optionally be a single-chain antibody fragment. Alternatively, the fragment may comprise multiple chains which are linked together, for instance, by disulfide linkages. The fragment may also optionally be a multi-molecular complex. A functional antibody fragment will typically comprise at least about 50 amino acids and more typically will comprise at least about 200 amino acids. As used herein, an antibody also includes bispecific and chimeric antibodies.

Naturally produced antibodies can be generated using well-known methods (see, e.g., Kohler and Milstein (1975) Nature 256:495-497; Harlow and Lane (1988) supra). Alternatively, antibodies which specifically recognize the phosphorylation state of kinesin-1 are derived by a phage display method. Methods of producing phage display antibodies are well-known in the art (e.g., Huse, et al. (1989) Science 246(4935):1275-81).

Selection of kinesin-1-specific antibodies is based on binding affinity to kinesin-1 which is either phosphorylated or unphosphorylated at serine 176 (kinesin-1A or kinesin-1C) or serine 175 (kinesin-1B) and can be determined by the various well-known immunoassays indicated above.

The invention is described in greater detail by the following non-limiting examples.

### Example 1

### Materials and Methods

Antibodies and Reagents. The following antibodies were used: H2 and 63-90 monoclonal antibody anti-KHC (Stenoien & Brady (1997) Mol. Biol. Cell 8:675-689); androgen receptor (N-20; Santa Cruz Biochemicals, Santa Cruz, Calif.); tubulin antibody (Clone DM1a; Sigma, St. Louis, Mo.); phosphorylation-sensitive NF antibodies (SMI31 and SMI32; Sternberger Inc., Baltimore, Md.); GSK-3 (011-A) and dynein heavy chain (R-325) from Santa Cruz Biochemicals; GAP-43 (Boehringer Mannheim, Germany); Akt (05-591) and JNK (06-748) from Upstate Biotechnology (Lake Placid, N.Y.); and p38 (9217; Cell Signaling Technology, Inc., Danvers, Mass.).

SB203580, SP600125, okadaic acid, and JIP peptide (JNK inhibitor I; #420116) were from Calbiochem (San Diego, Calif.). Inhibitor stocks were in DMSO and stored in aliquots at −80° C. until used. Recombinant 6-His-tagged JNK3 kinase was from Upstate Biotechnology and CREB phosphopeptide from New England Biolabs (Ipswich, Mass.). GST-cJun (1-89) is known in the art.

Lysate Preparation/Immunoblot Analysis. Cell cultures were homogenized in ROLB buffer (10 mM HEPES pH 7.4, 0.5% TRITON X-100, 80 mM β-glycerophosphate, 50 mM sodium fluoride, 2 mM sodium orthovanadate, 100 nM staurosporine, 100 nM K252a, 50 nM okadaic acid, 50 nM microcystin, 100 mM potassium phosphate and mammalian protease inhibitor cocktail (Sigma)). Lysates were clarified by centrifugation and protein concentration determined using BCA kit (Pierce Biotechnology, Rockford, Ill.). Proteins were separated by SDS-PAGE and immunoblotted according to known methods (Morfini, et al. (2004) EMBO J. 23:2235-2245).

Fractionation of Cells Expressing Wild-Type Androgen Receptor and PolyQ-AR. Four 70% confluent 100-mm culture dishes containing either wild-type or 902-6 SH-SY5Y cells were homogenized in ROLB buffer. Lysates were centrifuged at 150,000 g for 30 minutes at 4° C. Supernatants represent the detergent-soluble fraction. Pellets were resuspended in 10% SDS and solubilized pellets passed 8-10 times through a 30 gauge hypodermic needle to shear DNA. Aliquots from both fractions were analyzed by quantitative immunoblot. Binding of kinesin-1 to membranes was evaluated according to established methods (Morfini, et al. (2002) supra; Morfini, et al. (2000) In Kinesin Protocols, I. Vernos, ed., Humana Press, Totowa, N.J., pg. 147-162).

Microtubule Binding Assays. Microtubule binding assays was performed according to established methods (Stenoien & Brady (1997) supra). Briefly, three 70% confluent 100-mm culture dishes containing either wild-type or 902-6 SH-SY5Y cells were homogenized with 500 μl of HEM buffer (50 mM HEPES, 1 mM EGTA, 2 mM MgSO₄, 1% TRITON X-100, pH 7.2, 1 μM staurosporine, 1 μM K252a, 50 nM okadaic acid and 1/100 mammalian protease inhibitor cocktail (Sigma) at 4° C. Lysates were centrifuged at 50000 rpm for 5 minutes at 4° C.

Six hundred μg of total protein from clarified wild-type or 902-6 SH-SY5Y cells were incubated with 0.2 mg of TAXOL-stabilized microtubules (Cytoskeleton, Denver, Colo.) and 2.5 mM AMP-PNP for 30 minutes at 37° C. Samples were centrifuged for 25 minutes at 50000 rpm (4° C.) over a cushion of 20% sucrose in HEM plus 20 μM TAXOL. Pellets and supernatants were collected and the amount of kinesin in each fraction was assayed by immunoblot with anti-kinesin monoclonal antibodies.

SH-SY5Y Cell Culture and Pharmacological Inhibition. Androgen receptor constructs containing wild-type androgen receptor (Q20) and pathogenic androgen receptor (Q56) tracts were prepared and stably transfected into SH-SY5Y human neuroblastoma cells (Avila, et al. (2003) Exp. Biol. Med. (Maywood) 228:982-90). Wild-type and 902-6 cells (expressing both full-length polyQ-AR and a truncated N-terminal androgen receptor fragment (≈25 kD) that accumulated in cytoplasm) were grown and differentiated (Szebenyi, et al. (2003) supra). Cells were plated at densities of 10,000 cells/cm² on 10-cm tissue culture dishes for biochemical studies, and at 3000-5000 cells/cm² on 4-well tissue-tech chamber slides (Becton-Dickinson, Mountain View, Calif.) for immunocytochemistry and morphometric studies. For inhibitor studies, cells were differentiated with 10 μM retinoic acid in serum for 5-6 days, and then switched to serum-free medium supplemented with 25 ng/ml BDNF (Alomone Laboratories, Jerusalem, Israel) with or without SB203580 inhibitor. Cell morphologies were evaluated after 3 days in BDNF±SB203580 (Szebenyi, et al. (2003) supra).

Recombinant Polypeptides. Wild-type androgen receptor and polyQ-AR polypeptides were produced by in vitro transcription/translation (TNT T7-Coupled Reticulocyte Lysate System; Promega, Madison, Wis.) according to manufacturer's protocols. Typically, 1.8 μg of plasmid was transcribed in 50 μl reaction mix. To assess protein levels, parallel reactions were performed incorporating ³⁵S-labeled methionine (Amersham) or quantitative immunoblots were performed. Protein concentrations were typically 0.2-0.5 nM. In vitro translated androgen receptor has been characterized and shown to be functional (Kuiper, et al. (1993) Biochem. J. 296(Pt 1):161-7). In vitro translation products were briefly centrifuged to eliminate translation machinery, and supernatants frozen in liquid N₂ until used.

Genes encoding human wild-type Huntingtin (Htt) (i.e., Q20, residues 1-548; Szebenyi, et al. (2003) supra) and polyQ-expanded Htt (i.e., Q46, residues 1-949; Qin, et al. (2004) J. Neurosci. 24(1):269-81) were cloned into the pcDNA.3 vector and produced by in vitro transcription/translation as described for the androgen receptor. Alternatively, shorter constructs (Htt exon1) were expressed in E. coli as GST-tagged proteins. All constructs above in their polyQ-expanded versions similarly inhibited fast axonal transport when perfused isolated squid axoplasm.

Kinesin-1 ATPase Assays. Kinesin-1 basal and microtubule-activated ATPase activity was assayed according to known methods (Morfini, et al. (2002) supra; Tsai, et al. (2000) Mol. Biol. Cell 11:2161-2173). Briefly, purified rat brain kinesin and in vitro-translated androgen receptor constructs were incubated with or without TAXOL-stabilized MAP-free microtubules (1 mg/ml). Assays were started by addition of 1 mCi γ-³²P ATP (ICN Biochemicals, Costa Mesa, Calif.) and incubated for 25 minutes at 37° C. Reactions were stopped with 10% SDS and aliquots spotted on PEI-cellulose plates. Chromatograms were developed in 0.5 M LiCl/1 M formic acid and spots for ³²P and γ-³²P ATP counted to obtain percent of total ³²P recovered as free phosphate.

Squid Axoplasm. Isolated squid axoplasm represents a unique experimental system to evaluate axonal-specific effects and pathogenic mechanisms. This model was instrumental in the original discovery of kinesin-1 (Brady (1985) Nature 317:73-75, novel pathways for fast axonal transport (Morfini, et al. (2002) supra; Morfini, et al. (2004) supra), and axonal-specific phosphorylation events (Grant & Pant (2000) J. Neurocytol. 29:843-72). Bidirectional membrane-bound organelle movements are observed with properties unchanged from intact axons for hours after removal of plasma membrane (Brady, et al. (1982) Science 218:1129-1131). Video-enhanced microscopic techniques allow quantitative analysis of membrane-bound organelle movement in fast axonal transport. Typical kinesin-dependent transport rates are 1.5-2.0 μm/s, whereas retrograde, cytoplasmic dynein-dependent rates are 1-1.3 μm/s in perfused axoplasms. These rates are maintained with little (<10%) or no reduction for >1 hour after perfusion with control buffer (Brady, et al. (1982) supra). The lack of permeability barriers allows perfusion of the axoplasm with a variety of effector molecules at known concentrations. Effectors of interest include nucleotides, pharmacological inhibitors, recombinant polypeptides, and antibodies. Typically, 6-His, Hemagglutinin, Myc and GST-tagged recombinant proteins are either expressed in bacteria or in vitro translated, purified and perfused (Szebenyi, et al. (2003) supra; Morfini, et al. (2002) supra).

To date, every observation made in squid axoplasm has been subsequently confirmed in mammalian models, starting from the original discovery of kinesin-1. Specific pathogenic effectors associated with neurodegeneration in Alzheimer's (i.e., filamentous and soluble Tau, Abeta oligomers, Huntington's (polyQ expanded huntingtin (Szebenyi, et al. (2003) supra)), Kennedy's (polyQ-expanded androgen receptor) and Parkinson's (mutant a-synuclein, Lewy filaments and MPP+) diseases as well as Amyotrophic Lateral Sclerosis (mutant SOD1) have been examined in the squid system. In each case, specific changes in fast axonal transport associated with these pathogenic proteins/compounds have been demonstrated, and in most cases been confirmed by parallel changes in signaling pathways and molecular motors in mammalian models of these same diseases.

Phosphorylation Studies in Squid Axoplasm. Three to four axoplasms were triturated in KB buffer (KB: 10 mM HEPES pH 7.4, 10 mM MgCl₂, 1 mM DTT) and aliquoted. Individual reactions with corresponding in vitro-translated androgen receptor constructs were started by adding radiolabeled ATP to 100 μM. Reactions were done in 50 μl, incubated for 20 minutes at room temperature and stopped with 50 μl of 2× sample buffer. Lysates were separated by SDS-PAGE and analyzed by autoradiography. For certain indicated assays, 2 axoplasms per tube were incubated in X/2 buffer for 50 minutes with corresponding in vitro-translated androgen receptor construct, then 50 μl of 2× sample buffer was added and samples analyzed by immunoblot.

Metabolic Labeling Experiments in Cells and Squid Axoplasm. SH-SY5Y cells were cultured and differentiated as above. After 5 days differentiation, 1 mCi ³²P phosphate (ICN Biochemicals) was added per dish and incubated for 4 hours. Media was discarded, cells homogenized in 1 ml of ROLB buffer and processed according to known methods (Morfini, et al. (2004) supra). For certain experiments, axoplasm were prepared as for video microscopy, but 1 mCi of γ-³²P-ATP was added. After 50 minutes incubation, axoplasms were homogenized in ROLB buffer. A 10 μl aliquot of lysate was precipitated with 15% TCA and radioactivity incorporated into protein determined by scintillation counting. Aliquots of equal counts were used for kinesin immunoprecipitation with 10 μg of H2 antibody and Protein G-agarose beads (Pierce Biotechnology). Immunoprecipitates were separated by SDS-PAGE, dried and exposed in a PHOSPHORIMAGER cassette, then scanned and quantified on a TYPHOON (Amersham/Molecular Dynamics, Sunnyvale, Calif.).

Motility Studies in Isolated Axoplasm. Axoplasm was extruded from giant axons of the squid Loligo pealeii (Marine Biological Laboratory, Woods Hole, Mass.) as described (Brady, et al. (1985) Cell Motil. 5:81-101). Axons were 400-600 μm in diameter and provided ≈5 μl of axoplasm. Recombinant androgen receptor constructs, JNK, peptides and inhibitors were diluted into X/2 buffer (175 mM potassium aspartate, 65 mM taurine, 35 mM betaine, 25 mM glycine, 10 mM HEPES, 6.5 mM MgCl₂, 5 mM EGTA, 1.5 mM CaCl₂, 0.5 mM glucose, pH 7.2) supplemented with 2-5 mM ATP and 20 μl of this mix was added to perfusion chambers (Brady, et al. (1985) supra). Preparations were analyzed on a ZEISS AXIOMAT with a 100×, 1.3 n.a. objective, and DIC optics. Hamamatsu Argus 20 and Model 2400 CCD camera were used for image processing and analysis. Organelle velocities were measured with a Photonics Microscopy C2117 video manipulator (Hamamatsu).

Immunoprecipitation Kinase Assays. Immunoprecipitation kinase assays were performed using 500 μg of total protein from differentiated SH-SY5Y cells according to known methods (Beffert, et al. (2002) J. Biol. Chem. 277:49958-49968). JNK was immunoprecipitated with 2 μg of each JNK1 (G151-333; Pharmingen, San Diego, Calif.) and SAPK1 (06-748; Upstate Biotechnology) antibodies. Control immunoprecipitates were carried out with 2 μg of each normal mouse or rabbit IgG. GST-cjun (1-89) (3 μg) was used as substrate. Reactions were carried out 20 minutes at 30° C., in the presence of 100 μM radiolabeled γ-³²P-ATP. Samples were analyzed by SDS-PAGE and gels were dried after staining with COOMASSIE Blue. Kinase activity values were obtained using a TYPHOON PHOSPHORIMAGER after overnight exposure. Background kinase activity values from immunoprecipitates with non-immune control antibodies were subtracted.

In Vitro Kinesin-1 Phosphorylation. A recombinant cDNA fragment coding for the first 584 amino acids of rat KHC (KHC-584) was subcloned into pET expression vector, expressed in E. coli and purified by nickel affinity chromatography (Qiagen, Valencia, Calif.). Aliquots of KHC-584 (10 μg) were incubated with 0.5 μg of recombinant JNK3/SAPK1b (Upstate Biotechnology) in 20 μl of HEM buffer (50 mM HEPES, 1 mM EGTA, 2 mM MgSO₄). Same reactions were performed using immunoprecipitated mouse brain kinesin (Morfini, et al. (2002) supra) as a substrate. Reactions were started by addition of 100 μM radiolabeled ATP. After 30 minutes at 37° C., reactions were stopped by adding 8 μl of 5× sample buffer. Proteins were separated by SDS-PAGE; the gels dried and exposed to a PHOSPHORIMAGER screen.

Immunocytochemistry. Immunocytochemical staining was performed in accordance with established methods (Szebenyi, et al. (2003) supra; Morfini, et al. (2002) supra). Briefly, cells were fixed for 15 minutes at 37° C. in 2% paraformaldehyde/0.01% glutaraldehyde/0.12 M sucrose in PHEM, washed in PBS and permeabilized with 0.2% TRITON X-100 in PBS for 10 minutes. Cultures were blocked for 1 hour in 2.5% gelatin/1% BSA in PBS and incubated overnight at 4° C. in a humid chamber with DM1a or N-20 primary antibodies followed by incubation with appropriate secondary antibodies conjugated with ALEXA Fluoro-red or Fluoro-green (Molecular Probes, Eugene, Oreg.). Fluorescence was visualized on a ZEISS LSM 510 confocal microscope or AXIOVERT 200M inverted microscope with OPENLAB image processing software. Primary antibodies were used at 0.5-5 μg/ml.

Cellular and Animal Models of Huntington's Disease. A mouse colony was established in which the endogenous copy of Htt (Q20) was replaced with polyQ-Htt (Q111), allowing polyQ-Htt expression at endogenous levels (Wheeler, et al. (2000) Hum. Mol. Genet. 9(4):503-513). Primary neuronal cultures from these animals are routinely prepared using established methods. Additionally, immortalized striatal cell lines derived from these animals are also available (Trettel, et al. (2000) Hum. Mol. Genet. 9(19):2799-2809). These models were specifically selected to avoid artifacts related to protein overexpression.

Statistical Analysis. All experiments were repeated at least 3 times. Unless otherwise stated, the data was analyzed by ANOVA followed by post-hoc Student-Newman-Keul's test in order to make all possible comparisons. Data was expressed as mean±SEM and significance was assessed at p<0.05 or 0.01 as noted.

### Example 2

### JNK Mediates Pathogenic Effects of Polyglutamine-Expanded Androgen Receptor on Fast Axonal Transport

PolyQ-AR inhibits fast axonal transport in isolated axoplasm (Szebenyi, et al. (2003) supra). Fast axonal transport in squid axoplasm depends on the activity of kinesin-1 motor proteins (Brady, et al. (1990) Proc. Nat. Acad. Sci. USA 87:1061-1065; Stenoien & Brady (1997) supra). Several studies have suggested that polyQ-expanded protein aggregates might inhibit fast axonal transport by directly binding and sequestering kinesin-1 (Gunawardena, et al. (2003) supra; Lee, et al. (2004) supra). It was predicted that kinesin-1 should partition with aggregates in fractionation of cells expressing polyQ-AR (Gunawardena, et al. (2003) supra). Therefore, cell lysates from stably transfected SH-SY5Y cells expressing wild-type androgen receptor or polyQ-AR (902-6) were fractionated to yield both detergent-soluble and insoluble fractions. Quantitative immunoblots for androgen receptor and kinesin-1 were used to calculate I/S ratios for androgen receptor and kinesin-1. The I/S ratio was 0.967 for wild-type androgen receptor and 1.83 for full-length polyQ-AR (n=3), although no aggregates were visible by light microscopy (Szebenyi, et al. (2003) supra; Avila, et al. (2003) supra). As noted (Szebenyi, et al. (2003) supra), a polyQ-AR fragment in 902-6 cells (which contains the polyQ tract) was almost entirely in the soluble fraction (I/S=0.043) (n=3). In contrast, I/S ratios for kinesin-1 were the same with both wild-type androgen receptor and pathogenic polyQ-AR-expressing cells (I/S=0.767; n=3). PolyQ-dependent changes in androgen receptor solubility did not affect kinesin-1 solubility. Thus, polyQ-AR effects on fast axonal transport and neurite outgrowth do not involve selective kinesin-1 binding and sequestration by polyQ-AR aggregates.

Studies have indicated that alterations in kinesin-1 binding to membrane cargoes can lead to inhibition of fast axonal transport (Morfini, et al. (2002) supra; Stenoien & Brady (1997) supra). Potential alterations in kinesin-1 membrane association and microtubule-binding were evaluated in three differentiated SH-SY5Y cell lines: one stably transfected with wild-type androgen receptor and two independent lines expressing polyQ-AR (902-6 or 902-13). These cell lines are known in the art (see Szebenyi, et al. (2003) supra). Total kinesin-1 levels were comparable in all cell lines. In subcellular fractionation experiments (Morfini, et al. (2002) supra), no differences were observed in the fraction of kinesin-1 binding to membranes between wild-type androgen receptor and polyQ-AR expressing cell lines.

Kinesin-1 functions also include binding to microtubules, and microtubule-activated ATPase activities. Kinesin-1 binding to microtubules in the presence of AMP-PNP was severely reduced in polyQ-AR-expressing cells, compared to untransfected cells and wild-type androgen receptor-expressing ones. In contrast, cytoplasmic dynein heavy chain (DHC) binding to microtubules was unaffected by expression of polyQ-AR. Kinesin-1 heavy chain/light chain stoichiometry was indistinguishable between Ctrl, wild-type androgen receptor and PolyQ-AR samples.

To determine whether polyQ-AR could affect kinesin microtubule-activated ATPase, ATPase activity was assayed with purified native kinesin-1 in the presence of wild-type androgen receptor or polyQ-AR and microtubule. Microtubule-activated ATPase activity of kinesin-1 was not affected by either wild-type androgen receptor or polyQ-AR. PolyQ-AR failed to affect either basal or microtubule-activated ATPase activity of kinesin-1 in vitro, even when assayed at equimolar levels of androgen receptor and kinesin-1. Taken together, these experiments indicated polyQ-AR induced specific alterations in kinesin-1 binding to microtubules through an indirect mechanism.

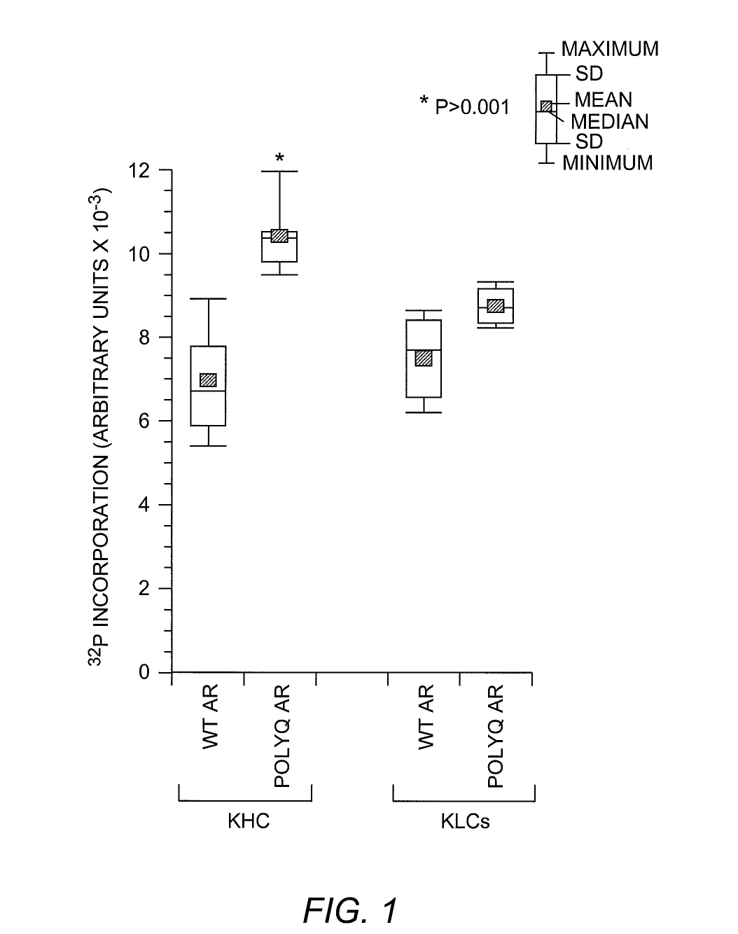

Phosphorylation is an indirect mechanism that can affect kinesin-1 function. Kinesin-1 motors are heterotetramers of two heavy (KHC) and two light (KLC) subunits (Bloom, et al. (1988) Biochemistry 27:3409-3416), and are regulated in vivo by phosphorylation (Morfini, et al. (2002) supra; Hollenbeck (1993) J. Neurochem. 60:2265-2275; Donelan, et al. (2002) J. Biol. Chem. 277:24232-24242). KHCs are responsible for microtubule-binding and ATPase hydrolysis, whereas KLCs mediate binding to specific membrane cargoes. Several kinases have been shown to phosphorylate specific kinesin-1 subunits, and to affect specific kinesin-1 functions (Morfini, et al. (2001) supra). Further, polyQ-AR expression was reported to activate kinase activities (LaFevre-Bernt, et al. (2003) supra). To determine effects of polyQ-AR on kinesin-1 phosphorylation in intact cells, differentiated SH-SY5Y cells stably transfected with wild-type androgen receptor or polyQ-AR were metabolically labeled with ³²P. Although total kinesin-1 levels were comparable in both cell lines, KHC phosphorylation increased by approximately 50% in cells with polyQ-AR, without significant changes in KLC phosphorylation (FIG. 1). KHC is the microtubule-binding subunit in kinesin-1, so a selective change in phosphorylation of KHC, but not KLC, induced by polyQ-AR is consistent with polyQ-AR-induced inhibition of kinesin-1 microtubule-binding activity.

To determine whether polyQ-AR activates kinase pathways in the absence of transcriptional changes, effects of polyQ-AR on the phosphorylation pattern of axonal proteins from isolated squid axoplasm, which lacks both nucleus and protein synthetic machinery, was evaluated. Multiple polypeptides exhibited increased incorporation of ³²P with polyQ-AR, including neurofilament (NF) subunits. Neurofilaments are the major phosphoproteins in axoplasm, being subject to phosphorylation by several protein kinases (Grant & Pant (2000) supra). Immunoblots with phosphosensitive antibodies against neurofilament KSP repeat domains showed that immunoreactivity with SMI32, which recognizes a dephosphorylated epitope in the KSP repeats, was reduced in axoplasms incubated with polyQ-AR, but not wild-type androgen receptor. SMI31 antibody immunoreactivity, which recognizes a different phosphorylated epitope in NFH, remained largely unaffected. SMI32 immunoreactivity changes were similar to those reported in an SBMA animal model (Chevalier-Larsen, et al. (2004) supra). These results indicate that polyQ-AR could induce changes in axonal kinase activities in a nuclear-independent manner and indicated that proline-dependent protein kinases were among the affected kinases.

These observations indicated that pathogenic polyQ-AR protein increased KHC phosphorylation through activation of one or more axonal phosphotransferase activities. Proline-dependent protein kinases involved in neurofilament phosphorylation include GSK-3, CDK5 (Bloom, et al. (1988) supra), and SAPKs (Grant & Pant (2000) J. Neurocytol. 29:843-872). Some of these kinases can affect kinesin-1-based motility (Morfini, et al. (2001) supra; Morfini, et al. (2002) supra; Morfini, et al. (2004) supra). Vesicle motility assays in isolated squid axoplasm were used to determine specific kinases responsible for polyQ-AR-induced fast axonal transport inhibition. GSK-3 is a neurofilament kinase that inhibits fast axonal transport by directly phosphorylating kinesin-1 (Morfini, et al. (2002) supra). To determine whether GSK-3 mediated polyQ-AR inhibition of kinesin-1-based motility, polyQ-AR was co-perfused with 0.5 mM CREBpp in isolated axoplasm. CREBpp is a GSK-3 peptide substrate that acts as a competitive inhibitor and blocks GSK-3-mediated inhibition of kinesin-based motility (Morfini, et al. (2002) supra; Morfini, et al. (2004) supra). However, CREBpp failed to prevent inhibition of fast axonal transport by polyQ-AR. Protein phosphatase activation can also affect kinesin-1-based motility (Donelan, et al. (2002) supra; Morfini, et al. (2004) supra). To test whether phosphatases contribute to polyQ-AR-induced inhibition of fast axonal transport, polyQ-AR was co-perfused with okadaic acid, a strong inhibitor of PP1 and PP2 serine-threonine phosphatases (Hardie, et al. (1991) Meth. Enzymol. 201:469-476). Okadaic acid blocks a CDK5-related pathway leading to inhibition of kinesin-1 (Morfini, et al. (2004) supra), but failed to prevent polyQ-AR-induced fast axonal transport inhibition.

Among the inhibitors and kinase substrates co-perfused initially, only GST-cJun (1-89) significantly attenuated polyQ-AR-induced inhibition of fast axonal transport. Perfusion of GST or GST-cJun (1-89) alone in axoplasm had no effect on fast axonal transport, indicating that cJun (1-89) acted as a competitive inhibitor of an endogenous kinase. GST-cJun (1-89) is a fusion protein that includes the first 89 amino acids of cJun protein and is a specific substrate for selected stress-activated protein kinases (SAPKs). GST-cJun at 50 μM protected fast axonal transport for the first 30 minutes when co-perfused with polyQ-AR, with a mean rate of 1.57±0.05 μm/sec with cJun as compared to 1.25±0.03 with polyQ-AR alone (significant at p≦0.001 in two sample t-test). However, fast axonal transport began to decline after 35-40 minutes with both cJun and polyQ-AR (mean rate of 1.29±0.03 μm/sec at 40-50 minutes, difference significant at p≦0.001 relative to the 20-30 minute rate with cjun). This value was comparable to polyQ-AR alone at 20-30 minutes, but still significantly higher that the rate seen with polyQ-AR alone at 40-50 minutes (1.08±0.02 μm/sec; difference significant at p≦0.001). In contrast, rates with wild-type androgen receptor were unchanged between 20-30 minutes (1.71±0.03 μm/sec) and 40-50 minutes (1.77±0.04 μm/sec). Given that CREBpp at 0.5-1 mM blocked GSK-3 effects on transport for >60 minutes (Morfini, et al. (2002) supra), these data indicated that available GST-cJun might become completely phosphorylated toward the end of these assays.

To confirm that SAPKs mediate inhibition of fast axonal transport by polyQ-AR, polyQ-AR was co-perfused with SB203580 (10 μM). SB203580 is a highly specific pharmacological kinase inhibitor of selected SAPKs, tested for more than 100 kinases (Fabian, et al. (2005) Nat. Biotechnol. 23:329-36). In co-perfusion experiments, SB203580 completely blocked the inhibition of fast axonal transport in both anterograde and retrograde directions by polyQ-AR. Collectively, these data indicated that polyQ-AR-induced inhibition of fast axonal transport depends upon activation of one or more SAPK.

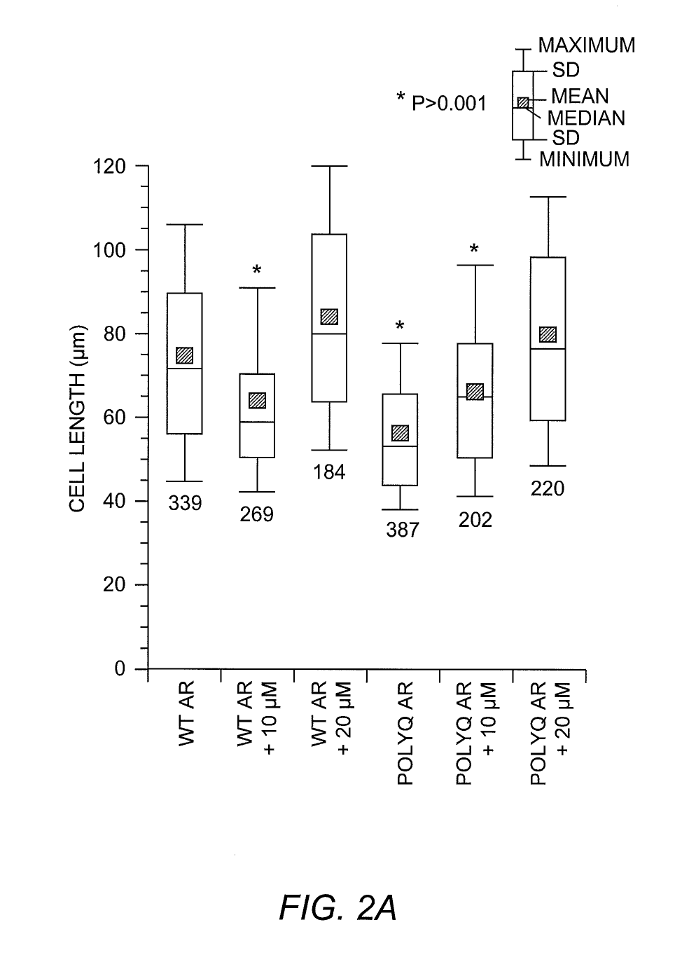

Sequential treatment with retinoic acid and BDNF induces SH-SY5Y cells to stop dividing, differentiate as neurons, and become dependent on BDNF for survival (Szebenyi, et al. (2003) supra). SH-SY5Y cells stably transfected with wild-type androgen receptor become spindle-shaped and extend long neurites, while most 902-6 cells (an SH-SY5Y cell line stably transfected with polyQ-AR) remain flat and polygonal (Szebenyi, et al. (2003) supra). The difference between untreated wild-type androgen receptor and 902-6 cells in total neurite length was significant at p<0.01 by ANOVA (FIG. 2A). However, addition of SB203580 to culture media overcame inhibition of neurite outgrowth by polyQ-AR (FIG. 2A and FIG. 2B). Quantitation of SH-SY5Y morphologies with and without SB203580 showed that both wild-type and 902-6 cells responded to treatment with 10 μM SB203580 by increasing neurite outgrowth. For wild-type androgen receptor cells, this may reflect normal modulation of neurite outgrowth by SAPK kinases. However, the effect of SB203580 on 902-6 cells was more pronounced than in wild-type androgen receptor cells (FIG. 2A). Histograms in FIG. 2B show changes in 902-6 cell shapes with SB203580 treatment. Cultures of untreated 902-6 included very few cells with processes >80 μm, whereas in cultures of SB203580-treated 902-6 cells, there was a dramatic and dose-dependent increase in the proportion of cells that extended long neurites (>80 μm in length). Morphology distributions were nearly indistinguishable between wild-type and 902-6 cells with 20 μM SB203580. These experiments were consistent with data from co-perfusion experiments, and indicated that SAPK activity also mediates inhibition of neurite outgrowth due to polyQ-AR.

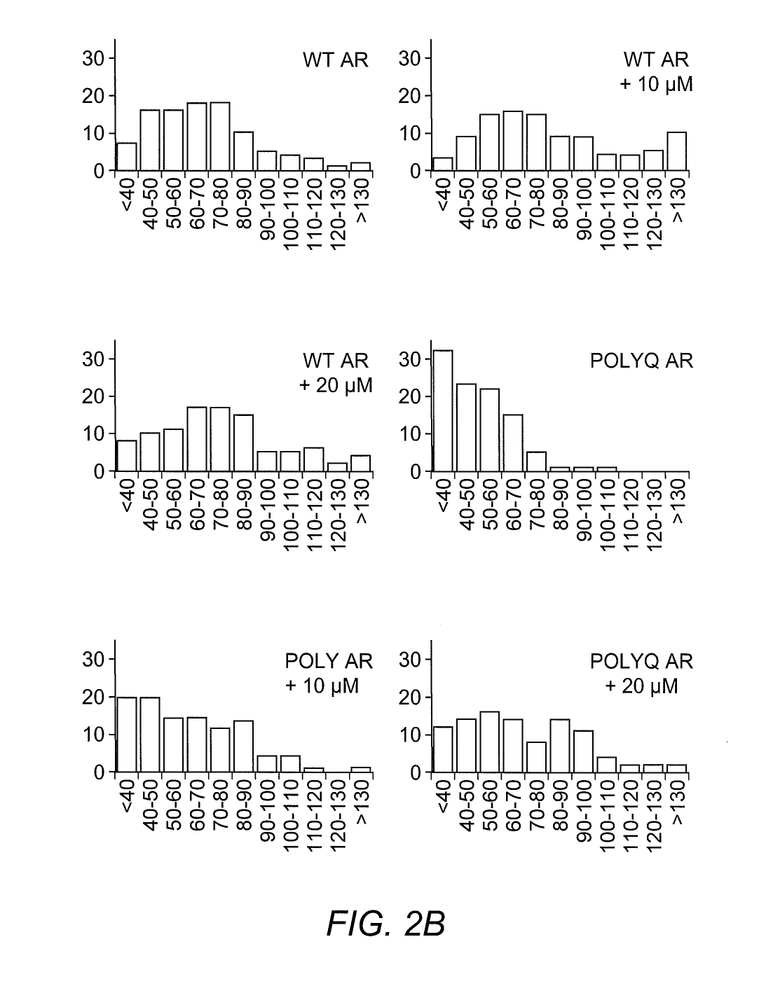

The specificity of SB203580 indicated that kinases mediating inhibition of fast axonal transport and polyQ-AR-induced neurite outgrowth were members of the p38/SAPK2 (Fabian, et al. (2005) supra), or JNK/SAPK1 (Coffey, et al. (2002) J. Neurosci. 22:4335-45) SAPK subfamilies. To gain insights on the SAPKs involved, expression of SAPK kinases was analyzed during SH-SY5Y differentiation. The results of this analysis showed that the expression profile of SAPKs throughout SH-SY5Y differentiation resembled the developmental expression profile of SAPKs from nervous tissue (Coffey, et al. (2000) J. Neurosci. 20:7602-13). As SH-SY5Y cells acquired a neuronal-like phenotype, p38 kinase levels were dramatically reduced. JNK protein levels, however, remained at relatively high levels, focusing attention on SAPK1/JNK kinases, rather than SAPK2/p38 kinases. To determine the effects of polyQ-AR on the activity of specific SAPKs, immunoprecipitation kinase assays were performed (FIG. 3). Consistent with immunoblots, p38/SAPK2 kinase activity was below detection limits in fully differentiated SH-SY5Y cells, but JNK/SAPK1 activity increased 3-fold in polyQ-AR-expressing cells relative to cells expressing wild-type androgen receptor.

Experiments above showed polyQ-AR increased JNK kinase activity, and KHC phosphorylation. To determine whether JNK kinase activation mediated polyQ-AR-induced fast axonal transport inhibition in axons, polyQ-AR was co-perfused with JNK kinase inhibitors in squid axoplasm. Co-perfusion of polyQ-AR with SP600125 (500 nM) restored kinesin-1-based motility. SP600125 was developed as an inhibitor of JNK and reported to show >20-fold selectivity for JNK over a wide range of protein kinases tested (Bennett, et al. (2001) Proc. Natl. Acad. Sci. USA 98:13681-6), including p38 kinases. Identical results were obtained when PolyQ-AR was co-perfused with JIP peptide (100 μM). JIP peptide contains a 20-amino acid inhibitory domain sequence derived from the JNK binding protein islet-brain (JIP1, IB), and inhibits JNKs, but not p38, with high specificity (Barr, et al. (2002) J. Biol. Chem. 277:10987-97). Moreover, polyQ-AR-induced changes in neurofilament phosphorylation were blocked by JIP peptide. Finally, recombinant active JNK kinase induced similar changes in SMI32 immunoreactivity, as did polyQ-AR.

Given that effects of polyQ-AR on fast axonal transport could be attenuated by co-perfusion of JNK inhibitors, perfusion of active JNK was expected to mimic polyQ-AR effects on fast axonal transport. Recombinant active JNK inhibited fast axonal transport in squid axoplasm and exhibited profile of inhibition similar to polyQ-AR. Further, immunoprecipitated kinesin-1 from JNK-perfused axoplasms showed increased KHC, but not KLC phosphorylation, consistent with results from metabolic labeling experiments in SH-SY5Y cells. These experiments indicated JNK kinase activity inhibits fast axonal transport through phosphorylation of KHC. Taken together, these data indicated axonal JNK kinase activation mediates polyQ-AR-induced neurofilament phosphorylation and fast axonal transport inhibition, in a nuclear and transcription-independent manner.

Results from JNK axoplasm perfusion led to the examination of whether JNK could directly phosphorylate kinesin-1. In vitro kinase assays showed that both recombinant KHC and immunoprecipitated endogenous mouse brain kinesin-1 KHC could be phosphorylated by recombinant JNK. JNK did not phosphorylate KLC in vitro, consistent with results from microtubule-binding assays, metabolic labeling, and axoplasm perfusion experiments disclosed herein. Together, these results indicated that KHC is a physiological JNK kinase substrate. JNK and polyQ-AR also have an effect on retrograde fast axonal transport, which raises the possibility of JNK may also have effects on cytoplasmic dynein.

### Example 3

### JNK Mediates Pathogenic Effects of Polyglutamine-Expanded Huntingtin on Fast Axonal Transport

Vesicle motility assays in isolated squid axoplasm were used to evaluate the effects of polyQ-expanded Htt on fast axonal transport. Perfusion of recombinant wild-type Htt in squid axoplasm showed no effect on either direction of fast axonal transport. As with polyQ-AR, perfusion of pathogenic, polyQ-Htt resulted in a striking inhibition of fast axonal transport rates (Szebenyi, et al. (2003) supra).

The polyQ-expanded polypeptides disclosed herein inhibited fast axonal transport at subnanomolar levels (≈0.5 nM), although kinesin-1 is present in axoplasm at a much higher concentration (≈500 nM). This indicated activation of enzymatic activities involved in fast axonal transport regulation. Consistently, PolyQ-Htt has been reported to activate multiple kinase/phosphatase pathways in several cellular models of Huntington's Disease (Wu, et al. (2002) J. Biol. Chem. 277(46):44208-13; Humbert, et al. (2002) Dev. Cell 2(6):831-7; Phelan, et al. (2001) J. Biol. Chem. 276(14):10801-10; Garcia, et al. (2004) Neuroscience 127(4):859-70).

To identify specific kinase activities responsible for inhibition of fast axonal transport by polyQ-Htt, axoplasms were co-perfused with polyQ-Htt, and specific peptide substrates or pharmacological inhibitors. As with polyQ-AR, SB203580 prevented the effects of polyQ-Htt on fast axonal transport. These findings are significant because common pathways for polyQ diseases have remained elusive (Morfini, et al. (2005) Trends Mol. Med. 11:64-70).

Data from co-perfusion experiments showed that SB203580 prevented the inhibitory effects of polyQ-Htt and polyQ-AR on fast axonal transport. Immunoblots and immunoprecipitation kinase assays indicated that both JNK and p38 kinases were present in squid axoplasm. Of these kinases, SB203580 inhibits p38α, p38β, JNK2, and JNK3. To identify SAPKs mediating polyQ-Htt-induced fast axonal transport inhibition, specific inhibitors of JNK were co-perfused with polyQ-Htt, and the effects analyzed using vesicle motility assays. Significantly, co-perfusion of JIP peptide (100 nM) along with polyQ-Htt prevented the inhibition of fast axonal transport induced by polyQ-Htt. JIP peptide contains a 20-amino acid inhibitory domain sequence derived from the JNK binding protein islet-brain (JIP1, IB) and inhibits JNKs, but not p38, with high specificity (Bonny, et al. (2001) Diabetes 50(1):77-82; Barr, et al. (2002) J. Biol. Chem. 277(13):10987-97). Taken together, these data indicate that, like polyQ-AR, JNK mediates polyQ-Htt-induced fast axonal transport inhibition.

As with other SAPKs, JNK activation involves phosphorylation by upstream mitogen-activated protein kinase (MAPKKs, typically MKK4 or MKK7), which phosphorylate JNK at the activation loop (threonine 183 and tyrosine 185 residues; Lawler, et al. (1998) Curr. Biol. 8(25):1387-90). The availability of antibodies against active forms of JNK allowed for the evaluation of JNK activity in vivo in a Huntington's disease mouse model. Striata from 14-month old wild-type, as well as heterozygous and homozygous Hdh^{Q109} CAG knock-in mouse brain were carefully dissected out, and processed for immunoblot analysis using phosphorylation-dependent anti-JNK antibody (pJNK Ab), which selectively detects dually phosphorylated, active JNK (Kujime, et al. (2000) J. Immunol. 164(6):3222-8).

Immunoblots showed comparable levels of JNKs expression among wild-type, heterozygous and homozygous mice, as revealed by a phosphorylation-independent JNK antibody. However, pJNK antibody showed a marked increase in JNK activation for mice expressing polyQ-Htt. Immunoblot analysis using recombinant, active JNK isoforms (JNK1, JNK2 and JNK3) revealed that the PJNK antibody used herein displayed similar affinity for all three JNK isoforms. Accordingly, several immunoreactive bands of variable molecular weight size were recognized by pJNK antibody, which correspond to various JNK gene products and isoforms (Gupta, et al. (1996) EMBO J. 15(11):2760-70). Notably, variable degrees of activation were observed of individual JNK isoforms. For example, a higher molecular band species recognized by pJNK antibody (p54) displayed a larger increase in immunoreactivity than a lower molecular species (p46). Consistent with dominant effects of polyQ-Htt, densitometric analysis of immunoblots revealed increased JNK activity in both heterozygous (100% of p54 band and 32% p46 band) and homozygous (160% p54 band and 62% p46 band) Huntington's disease mice compared with wild-type animals. Taken together, this data indicated that polyQ-Htt expression induces JNK kinase activation in vivo, consistent with results from co-perfusion experiments in squid axoplasm. In addition, quantitative analysis of JNK activation indicated differential activation of various JNK isoforms induced by polyQ-Htt expression.

Three JNK genes exit in mammals (JNK1, JNK2 and JNK3), which give rise to the alternative spliced isoforms (Gupta, et al. (1996) supra). JNK1 and JNK2 are ubiquitously expressed, whereas JNK3 is selectively expressed in neuronal cells (Mohit, et al. (1995) Neuron 14(1):67-78). The high degree of homology of the activation loop epitope among JNK isoforms does not allow the generation of phosphorylation-dependent antibodies that would recognize specific active JNK isoforms. Although the exact identity of JNK isoforms activated by polyQ-Htt was not determined, results of the analysis disclosed herein indicated different degrees of activation for different JNK isoforms. Therefore, it was determined whether specific JNK isoforms mediated the inhibition of fast axonal transport induced by polyQ-Htt.

The effects of recombinant, active JNK1, JNK2 and JNK3 proteins on fast axonal transport were directly evaluated using vesicle motility assays in squid axoplasm. The enzymatic activity of recombinant JNKs was first evaluated by in vitro kinase assays using c-Jun as a substrate. Perfusion of JNK1 at 200 nM concentration did not show any effect on either direction of fast axonal transport (FIG. 3). Unexpectedly, perfusion of JNK2 at 100 nM concentration slightly decreased anterograde, kinesin-1-dependent fast axonal transport rates (1.25 μM/sec), compared to 1.6 μM/sec mean anterograde fast axonal transport rates observed with control buffer (P≦0.01, two-sample t-test). Finally, perfusion of JNK3 dramatically inhibited anterograde fast axonal transport rates (mean rate for anterograde fast axonal transport was 0.9 μM/sec; P≦0.01 by two-sample t-test). This value was comparable to that seen with polyQ-Htt or polyQ-AR perfusion. In addition, JNK3 had an inhibitory effect on retrograde, cytoplasmic dynein-dependant fast axonal transport rates (1.25 μM/sec mean rate, compared to 1.4 μM/sec mean retrograde fast axonal transport rate observed with control buffer; P≦0.01, two-sample t-test), much like polyQ-Htt. These data indicated that the inhibitory effect of polyQ-Htt on fast axonal transport is mediated by the neuronal specific JNK3 isoform. Significantly, JNK3 is exclusively expressed in neuronal cells.

As described above, JNK kinases are regulated by phosphorylation. JNKs are substrates for MAPK kinases (MKKs), dual-specificity kinases that phosphorylate JNKs on both a threonine and a tyrosine residue in the activation loop of their catalytic domain (Lawler, et al. (1998) supra). This dual phosphorylation is absolutely required for activation of the JNKs. In addition, MKKs are also activated by phosphorylation within their activation loops. This is accomplished by a group of serine/threonine kinases known as the MAPK kinase kinases (MKKKs). Several MKKKs can activate the JNK pathway, including MEK kinases (MEKKs), apoptosis-inducing kinase 1 (ASK1) and transforming-growth factor beta (TGFβ)-activated kinase 1 (TAK1).

Based on observations from JNK activation in the Huntington's disease mouse model, it was determined whether MKKKs played a role in polyQ-Htt and polyQ-AR-induced inhibition of fast axonal transport. To this end, specific pharmacological inhibitors of various MKKKs were co-perfused with pathogenic Htt or androgen receptor in squid axoplasm. Notably, CEP-1347 prevented the effects of polyQ-Htt and polyQ-AR on fast axonal transport (FIG. 3). CEP-110024 is a highly specific pharmacological of MLKs, and does not inhibit other MAPKKKs. These data indicate that polyQ-expanded proteins inhibit fast axonal transport through a pathway involving MLK activation.

Perfusion experiments with all three recombinant JNK isoforms indicated that JNK3 mediates the inhibitory effect of polyQ-expanded proteins on fast axonal transport. Thus, it was determined whether JNK3 directly phosphorylates kinesin-1 using in vitro phosphorylation assays. Kinesin-1 exists as a heterotetramer of two KHCs and two KLCs. KHCs are responsible for microtubule-binding and ATPase hydrolysis, whereas KLCs mediate binding to specific membrane-bound organelles. To evaluate whether KHCs or KLCs represented a substrate for JNK3, immunoprecipitated endogenous mouse brain containing both KHCs and KLCs was phosphorylated with recombinant JNK3. Autoradiograms showed ³²P incorporation in KHCs, but not KLCs. To gain insights on functional consequences of JNK3 phosphorylation, a recombinant KHC construct encompassing the first 584 amino acids of KHC (KHC584) was phosphorylated. Phosphorylated KHC584 was excised from gels, and trypsinized for mass spectrometry analysis. Total tryptic digests were analyzed by MALDI-TOF mass spectrometry, and resulting masses were compared with the predicted tryptic digestion pattern of KHC584. In addition, the results were scanned to identify masses corresponding to the tryptic peptides shifted by multiples of 80 Da (the change in mass associated with an added phosphate group). A single tryptic peptide was identified with evidence of phosphorylation. This peptide, corresponding to amino acids 173 to 188 of KHC584, was present in both the native form, as well as in a form corresponding to a single phosphorylation event. No other evidence of phosphorylation was revealed through this analysis. A phosphorylated peptide within KHC motor domain (residues 1-350) was unequivocally identified by these studies, which encompassed two serine residues (serine 175 and 176). To map the site of phosphorylation in this peptide, the digestion products were separated in an Ion Trap MS instrument and both native and phosphorylated forms of the amino acids 173-188 peptide were analyzed by post-source decay (PSD), This analysis supported the identification of serine 176 as the phosphorylation site in this peptide (FIG. 4).

Results from in vitro phosphorylation experiments indicated that KHC represents a novel JNK3 substrate. Because KHCs are responsible for microtubule binding (Hirokawa, et al. (1989) Cell 56(5):867-78), it was contemplated that phosphorylation of KHCs by JNK3 might affect the ability of kinesin-1 to bind to microtubules. Therefore, the effects of polyQ-Htt expression on kinesin-1 binding to microtubules were determined using a cellular model.

As a first step, various cell lines were screened for JNK3 expression using antibody that specifically recognizes JNK3. Unexpectedly, a high variability in JNK3 expression was observed among different cell lines, including PC-12, N2a, SH-SY5Y cells and NSC34 cells. NSC34 is a hybrid cell line produced by fusion of motor neuron enriched, embryonic mouse spinal cord cells with mouse neuroblastoma (Salazar-Grueso, et al. (1991) Neuroreport 2(9):505-8), and these were found to express higher levels of JNK3. NSC 34 cells were transiently transfected with plasmid constructs containing the first 969 amino acid residues of Htt in either wild-type Htt (Q18) or polyQ-Htt (Q46) versions (Qin, et al. (2003) Hum. Mol. Genet. 12(24):3231-44).

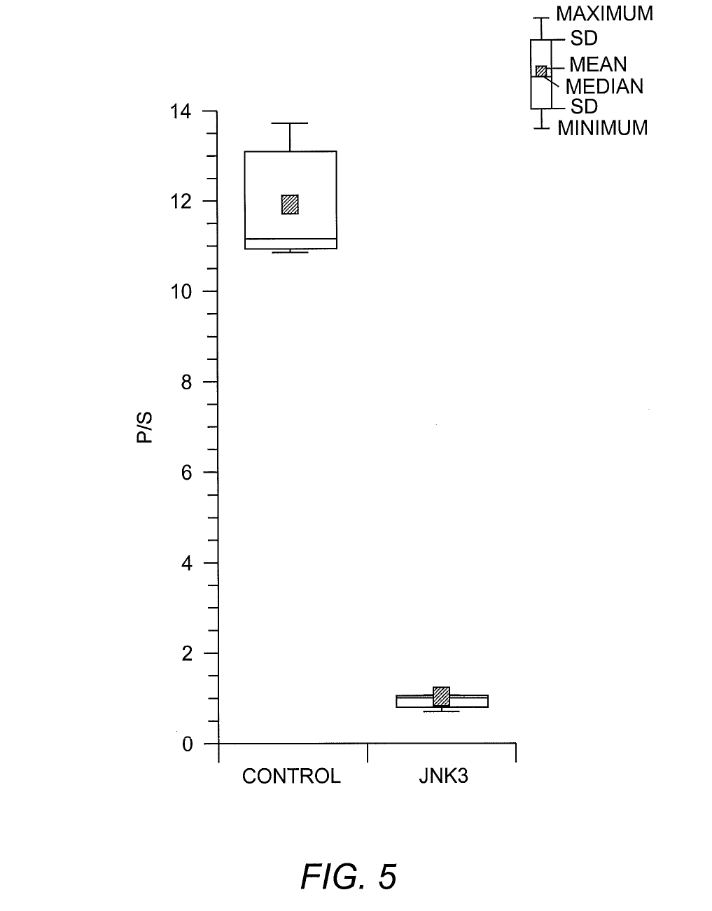

Microtubule-binding assays revealed that the binding of kinesin-1 to microtubules was severely reduced in polyQ-Htt-expressing cells, compared to untransfected or wild-type Htt-expressing ones. Total kinesin-1 levels were unchanged among untranfected, wild-type Htt, or polyQ-Htt-expressing cells. Taken together, results from these experiments indicated that polyQ-Htt expression significantly inhibited kinesin-1 binding to microtubules. These results were in agreement with findings showing reduction in the binding of kinesin-1 to microtubules elicited by expression of polyQ-AR expression. Moreover, treatment of kinesin-1 heavy chain with JNK3 kinase inhibits the binding of kinesin-1 to microtubules (FIG. 5).

What is claimed is:

1. A method for restoring fast axonal transport in a cell which expresses a polyglutamine-expanded polypeptide comprising contacting the cell with an effective amount of an agent which inhibits stress-activated protein kinase (SAPK)-dependent phosphorylation of kinesin thereby stimulating fast axonal transport in the cell.

2. The method of claim 1, wherein the kinesin is kinesin-1.

3. The method of claim 2, wherein the kinesin-1 is phosphorylated at serine 176 of SEQ ID NO:1 or SEQ ID NO:7, or serine 175 of SEQ ID NO:4.

4. The method of claim 1, wherein the SAPK is MLK3 or JNK3.

5. The method of claim 1, wherein the polyglutamine-expanded polypeptide is Huntingtin or androgen receptor.

6. A method for treating a polyglutamine expansion disease comprising administering to a subject with a polyglutamine expansion disease an effective amount of an agent which inhibits SAPK-dependent phosphorylation of a kinesin thereby treating the polyglutamine expansion disease.

7. The method of claim 6, wherein the kinesin is kinesin-1.

8. The method of claim 7, wherein the kinesin-1 is phosphorylated at serine 176 of SEQ ID NO:1 or SEQ ID NO:7, or serine 175 of SEQ ID NO:4.

9. The method of claim 6, wherein the SAPK is MLK3 or JNK3.

10. The method of claim 6, wherein the polyglutamine expansion disease is Huntington's disease, or spinal and bulbar muscular atrophy.

11. A method for identifying an agent for treating a polyglutamine expansion disease comprising contacting a SAPK with a test agent in the presence of a kinesin, or substrate fragment thereof, and determining whether the test agent inhibits the phosphorylation of the kinesin or substrate fragment by the SAPK thereby identifying an agent for treating a polyglutamine expansion disease.

12. The method of claim 11, wherein the kinesin is kinesin-1.

13. The method of claim 12, wherein the kinesin-1 is phosphorylated at serine 176 of SEQ ID NO:1 or SEQ ID NO:7, or serine 175 of SEQ ID NO:4.

14. The method of claim 11, wherein the SAPK is MLK3 or JNK3.

15. A method for monitoring treatment of a polyglutamine expansion disease comprising determining, in a biological sample from a subject receiving therapy for a polyglutamine expansion disease, the phosphorylation state of kinesin-1, wherein a decrease in the phosphorylation of kinesin-1 after receiving therapy is indicative of treatment of the polyglutamine expansion disease.
